SEQUENCE LISTING (6 sequences)

SEQ ID NO: 1 - DNA, 3336 bp, Trypanosoma brucei brucei
atgtcggatgccgatgtgaaagagggaacggcggccggcgattcagtggccgttcccgagtcggttgtaaaaccagatgaaggacggcggagcagaggtgagtctactggcgggacagctgctggggataccggtgtgccaaagaatatagcacggtgtcttgtttattgcaggttgaggccacggaacaagactgattttaagaacggtgggttccaactagtgacagtaagcgggaatgatattgttgtgaaggatcaacgcttttacaagtttgatggtgcttttggcgacgaatgtacacaaagtgatatatttgaagcggtggccgtcccttgcataacacacgcatttaaaggtttttgctcagcgttgatgtgctacggacagacgggtacaggtaagtctttcactatgtgtaataccactcctggccaagaaggcatcattccacggtccgccaaacttattttcgacaaaattcaatcagacaatgcgcggagttatgaagtgacaggacagtttgttcagatttaccgtgacaaccttggtgacttgatgagtgcaactggaagggaccgagtggatattcacttcgacgaacaagggggcgtagaacttaccggttgcagctcccatgttcttctgagtgcccaagagtttatgcgcttttaccgcatcggcaatgaccgtcgggttgtaactgcgactgctatgaatccggagtccagccgcggccatacagctttagttctccgcatcgtatcagagagccccagcgacccagaggcaggtaaactgaagggaaagattacattcatcgacttagcaggatacgagcgttttagtaaaactggtattacacatgacaaccccattatgaaggatgaggcgaagtgcatcaacgcctctcttctttcacttggtcacgttgtgtcgtgtttgtcgtcaggtagccggcacattccttggcgtgattcgaagctgacgcggatcctgcaggactctattggcggaaggagccgtacctctattattttgactgttgggccaagtagtgatcacctccacgaaaccacaaattcactgcagtttggtttgcgagcaatggatgtgaaggtgacggccaaacagtcggttcatgtggattaccagaagctggcccagaagctgcaatcactcttggatgaaagggacgagagaatcaatttactcgaagtgcagatcgcttctcgtgacgcagaaagacacgagttaatggagcgttacaacgatcgccgggaagacattgacagacgttttgagattgagatggctgaactgaagagaactggtgcatcggaagagcagatgctgaacctgcgtgaagtatacaaggctgaggtggaaaacctccaggagcagcaagacgaggagttccaatacagggaggaagtgtattcaaaggagatcgtccaccttattcgcgagcaggagcatcaggaagcgaagcgacgggcagagatgaaattggcgcaagatcttatcattgcggagttccaaaagaagctcgacaacgcgcgtgagggaacaaatgatgatctcgtcagagttttgaagcaactgtccgaaaaggacgccatattggccagccgagcgaacgacacggtgagactccacgaacatattgaggtgctcagggagcaagtgaaggagctcggtggagtgcctatagaggaggcgacgtttcccgaaacctttctggacgttggccaggtggaggagatgcggaaccggctggaggcggatgtgcaacgccatcgtgctaagggtgtggaattgcttgcggaagtggatcgtctttcgcagctctgctctgagcggttggaggagataaaccgactccgcgacgaaaacacacaatatcgcgccgcattggaaaacagtggcatttcattgaatgacactgatgatttgacggaattcctttctgagaagcgcactcagatggtggatgtttctgagatggaaactcttcgtgtcaccatgcaggccgaccttgatgaagcgaaggcgcacaaccgggagctggcgcgggaggtggagcagttgaagtttgaattaaccgcaaccgctattccactcacagcccggcttcgatgtccgccgtgcgcaactgcacgaggtccttccccgtttgacgccgcgcgcaacctgtgttcgacgcagcgtaaaccacctcaaaaggatggcacgccatccccaaacaacactcaaaatgaaaacttgcaaaggaccgtgaagcagcttacggagcaactggaattcagcatgcgtgagaggaagtcgcttcaggaccgcgttgaggctgttgagacgcaacttgcttcgcatggtgttgaggttccggggccgtacgtacccccaatcaaacttggtttccccggctctgcaccagtgacgtcatcggaaacagatgcaagggagccaccggaggataccgatatggatgtgctgctccgtgtaaaagaggaggaaatcgatgtgttattggaaacaattgaacggcaggagcacttgctcaatgctgcgaggtcgaatgaagagtttcaccgacgcgtcatttgtgagttgcagcagcagatggtgactgcgcaaatccaggtggaagatcctcagaacgcccctcctcctgttgacgccattgcaatggatgagtatatgtcaattttgcgtttagttcgggagtccgaacgcaagttggcagctcaattggctgagcgcgatggagaggatggcgcggaggtggaggccctgttggagaagaaggatgcggaactacaaatgaaggaggagaccatactcgagaaggcgtcgaaggcgcagtatgcagcgaagctctgcattcgtctgaagaaccagatgctgcgttgtggcatcacaccgtgttgtgagcttccagactcgtataacgagttgatcgagcgcgaagaggaggaactgaatgagcaactaatgtgccaagatgaactgttagccaggcttcgttcggaggaggaagaaaagcatcgcatgcagaatatgctgaaatcacttaatgaggagcgcgagaggcaatccagcgtcattcgaactgttcaagagcgctgtgaactggtggaaaagaaacaattggttacggcagcccacttgtcgcgattggcaacggaaaaatcccagagggagcaaattcttgaggaaacgctacgacgtgcaacacaagaattgttggattgcaagattaagatggccatggaaaaagaagcaggtagcccgggtgtgttaaagcgtttcctccgccgcctgcgctccaactga

SEQ ID NO: 2 - protein, 1111 aa, Trypanosoma brucei brucei
MSDADVKEGTAAGDSVAVPESVVKPDEGRRSRGESTGGTAAGDTGVPKNIARCLVYCRLRPRNKTDFKNGGFQLVTVSGNDIVVKDQRFYKFDGAFGDECTQSDIFEAVAVPCITHAFKGFCSALMCYGQTGTGKSFTMCNTTPGQEGIIPRSAKLIFDKIQSDNARSYEVTGQFVQIYRDNLGDLMSATGRDRVDIHFDEQGGVELTGCSSHVLLSAQEFMRFYRIGNDRRVVTATAMNPESSRGHTALVLRIVSESPSDPEAGKLKGKITFIDLAGYERFSKTGITHDNPIMKDEAKCINASLLSLGHVVSCLSSGSRHIPWRDSKLTRILQDSIGGRSRTSIILTVGPSSDHLHETTNSLQFGLRAMDVKVTAKQSVHVDYQKLAQKLQSLLDERDERINLLEVQIASRDAERHELMERYNDRREDIDRRFEIEMAELKRTGASEEQMLNLREVYKAEVENLQEQQDEEFQYREEVYSKEIVHLIREQEHQEAKRRAEMKLAQDLIIAEFQKKLDNAREGTNDDLVRVLKQLSEKDAILASRANDTVRLHEHIEVLREQVKELGGVPIEEATFPETFLDVGQVEEMRNRLEADVQRHRAKGVELLAEVDRLSQLCSERLEEINRLRDENTQYRAALENSGISLNDTDDLTEFLSEKRTQMVDVSEMETLRVTMQADLDEAKAHNRELAREVEQLKFELTATAIPLTARLRCPPCATARGPSPFDAARNLCSTQRKPPQKDGTPSPNNTQNENLQRTVKQLTEQLEFSMRERKSLQDRVEAVETQLASHGVEVPGPYVPPIKLGFPGSAPVTSSETDAREPPEDTDMDVLLRVKEEEIDVLLETIERQEHLLNAARSNEEFHRRVICELQQQMVTAQIQVEDPQNAPPPVDAIAMDEYMSILRLVRESERKLAAQLAERDGEDGAEVEALLEKKDAELQMKEETILEKASKAQYAAKLCIRLKNQMLRCGITPCCELPDSYNELIEREEEELNEQLMCQDELLARLRSEEEEKHRMQNMLKSLNEERERQSSVIRTVQERCELVEKKQLVTAAHLSRLATEKSQREQILEETLRRATQELLDCKIKMAMEKEAGSPGVLKRFLRRLRSN

SEQ ID NO: 3 - protein, 1111 aa, Trypanosoma brucei gambiense
MSDADVKEGTAAGDSVAVPESVVKPDEGRRSRGESTGGTAAGDTGVPKNIARCLVYCRLRPRNKTDFKNGGFQLVTVSGNDIVVKDQRFYKFDGAFGDECTQSDIFEAVAVPCITHAFKGFCSALMCYGQTGTGKSFTMCNTTPGQEGIIPRSAKLIFDKIQSDNARSYEVTGQFVQIYRDNLGDLMSATGRDRVDIHFDEQGGVELTGCSSHVLLSAQEFMRFYRIGNDRRVVTATAMNPESSRGHTALVLRIVSESPSDPEAGKLKGKITFIDLAGYERFSKTGITHDNPIMKDEAKCINASLLSLGHVVSCLSSGSRHIPWRDSKLTRILQDSIGGRSRTSIILTVGPSSDHLHETTNSLQFGLRAMDVKVTAKQSVHVDYQKLAQKLQSLLDERDERINLLEVQIASRDAERHELMERYNDRREDIDRRFEIEMAELKRTGASEEQMLNLREVYKAEVENLQEQQDEEFQYREEVYSKEIVHLIREQEHQEAKRRAEMKLAQDLIIAEFQKKLDNAREGTNDDLVRVLKQLSEKDAILASRANDTVRLHEHIEVLREQVKELGGVPIEEATFPETFLDVGQVEEMRNRLEADVQRHRAKGVELLAEVDRLSQLCSERLEEINRLRDENTQYRAALENSGISLNDTDDLTEFLSEKRTQMVDVSEMETLRVTMQADLDEAKAHNRELAREVEQLKFELTATAIPLTARLRCPPCATARGPSPFDAARNLCLTQRKPPQKDGTPSPNNTQNENLQRTVKQLTEQLEFSMRERKSLQDRVEAVETQLASHGVEVPGPYVPPIKLGFPGSAPVTSSETDAMEPPEDTDMDVLLRVKEEEIDVLLETIERQEHLLNAARSNEEFHRRVICELQQQMVTAQIQVEDPQNAPPPVDAIAMDEYMSILRLVRESERKLAAQLAERDGEDGAEVEALLEKKDAELQMKEETILEKASKAQYAAKLCIRLKNQMLRCGITPCCELPDSYNELIEREEEELNEQLMCQDELLARLRSEEEEKHRMQNMLKSLNEERERQSSVIRTVQERCELVEKKQLVTAAHLSRLATEKSQREQILEETLRRATQELLDCKIKMAMEKEAGSPGVLKRFLRRLRSN

SEQ ID NO: 4 - protein, 1102 aa, Trypanosoma congolense
MLADEATSTAGAAPSPPHAPSPSASKEAAAPAEGEGAPKSLSRCLVYCRLRPRNKTDFKNGGFQLLSVNGNDIVVKDQRFYKFDGSFNDDCTQSDIFETVAMPCITHAFKGFCAALMCYGQTGTGKSFTMCNTVPGQEGIIPRAAKFIFDSIESDSTKSYEVVAQFVQIYRDNLGDLMSSTGRDRVDIHFDETVGVELTGCTSHVLNSAQEFMRFYRIGNERRVVTATAMNPESSRGHTALVIRIFSENLNDPEVGKLKGKITFIDLAGYERFSKTGITHDNPIMKDEAKCINASLLSLGHVVSCLSSGSRHIPWRDSKLTRILQDSIGGRSRTSIILTVGPSSDHLHETTNTLQFGLRAMDVKVRAKQSVHVDYRKLAQKLQSLLNEKDEKINLLEVQIASRDAERQELMDRYSDRREDIERRFEKEMAELRQTGASEEQMCNLREVYKAEVENLNEQQDEEIQYKEEEYSKEIAKLVREQEHQEAKRRAEMKLAQELIIDDFQKKLESARGGTNDDLVRVLQQLAEKDAILASRANDTVRLHAHIEVLTEQVKQLGGVPVGEAMFPETFLDVGQVEEMQRRLEAEVERHHLKGVELRAEVDRLSQLCTERLEEINKLCDENSQFRAILEASGVAISDTDELTEFLRDKRTQMVDFAEMETLRVTMQADLDEVKAHNRELLREIERLKDEAATAHVPLTARLRGPTCFTARCPPSFDPMFKLFGGQNVSARGGATPPNSARRESMQKTVKKLSEKLESSMREKKTLIERVAAAEAELVLHGVEAPAPYVPPIKLDFPRELTASVEDDNEEDPLGNTDMDVLLRAKDDEIDSLLETIERQEYLLSTARSNEEFHRHVILELQQRLVAANIEIEENQNALPPVEGIAMDEYMLILRTLRESERKLAVQLADRDGQDSVEVDALLEGKDKELHQLEESVLEKASKVQFVAKMCIRLKNQMERLGIVPCCELPDSYRELIEREEDEINEQLESQEELMAKLRFEEEEKQRMQNMLKSLNEERERQSSVIRSVQERCKEVEKKHLDTAAHLSRLTQEKSQRELILEETLRRATQELLDCKIKMAMNKETNSTPGVFRRFLRRLRPG

SEQ ID NO: 5 - protein, 1107 aa, Trypanosoma vivax
MTDQVAAVQTVASPLVAPKGEDGRSQSKGGARAVPASGVATVEGDEPKNVSRCLVYCRLRPRNKTDFKNGGFQLVTVSENNVIVKDQRYYKFDGAFNEDCTQENIFDAVAVPCVTHAFKGFCSALMCYGQTGTGKSFTMCNTTPGSEGIIPRTAKFIFDTVEADRSRSYEVIGQFVQIYRDNLGDLMVGNGKDRVDIHFDDEGGVDLTGCSSHVLKSAQEFMRFYNSGNDRRVVTATAMNPESSRGHTALILRIISESVDDPASGKLKGKITFIDLAGYERFSKTGITHDNPIMKDEAKCINASLLSLGHVVSCLSSGSRHIPWRDSKLTRILQDSIGGRSRTSIILTVGPSSDHLHETTNSLQFGLRAMDVKVTAKQSVHVDYEKLARKLQLLLEEKDEKINFLEVQIASQDAERQELLEMYNAHRKAIDQRFENDMAQLTKTGASEQQILNLREVYKAEVENLREQQDEDILYREEEYSKRISKLVRDQVRQEEKRRAEMKLAQERIIEDFQKKLDKAREGTNDDLVKALQQLAEKDSILASRANDTARLHEHIEVLTEQVKELGGVPVEEATFPETFLDVGQVEEMQMQLEAEVERHRSKGIGLRAEVDRLSHLCTERLEEINKLNRENSQLREVLRESGIAIEDTDEVEQFMREARTRMIDISEMETLRVTMQEDLNEVKAHNRELEREVKRLRDELSTKAVPLTARIHRGTCGFGPNTVRGLGSTQTFSRTQHGLYTPPPSKPLEDSQRFVKKLSNQLEFSMREKNSLQERVTALEAELANAWPGDPAAICTPHHAPAIFFFYSLARRDEDNEPPTGKDVDVLLQVKDDEIDSLLETIEQQEFMLNTARSNDEFQKQLICELQQQVVAAKLDVKEHKTLPPPVDSISISDYMCLLRTIRDSERKLTTQLAERDGRDPVELDALLEKRDKELLLKDEAIVEKASKAQFVAKVCIRLKNQMERLGITPCCRLPDSYNELIEQEVCELENQVETQRELEERLRLEEEEKQRMANMLQSLKEERERQANVIRSVQARCREVEEKEMATAAHLSRLAKEKSQRELILEDTLRRATQELIESRVRLAMAEEVVESGMFNRLIRRWKQR

SEQ ID NO: 6 - protein, 1117 aa, Trypanosoma cruzi
MADQGVVAAPTDNAVKEAADLTPAQRVENFKDQKKALNKSTNDVPQPVNRCLVYCRLRPGNKTDFKEGSFKLVSVEGNSIVLKGERRYDFDKSFDDECTQEQIFDHVAVPCVDHAFNGFCSALMCYGQTGTGKSFTMCNTTPGLEGIIPRSARLIFERIQADPTRQYEVIGQFVQIYRDHLGDLMVGSGKERVEVRFDENEGVELTGCTSHVLRSPQEFMRFYHVGNERRVVTATAMNPESSRGHTALMIRIFSEKLDDPAAGKMRGKITFIDLAGYERFSKTGISSDNPIMKDEAKCINASLLSLGHVVTALSSSGPHIPWRNSKLTRILQDSIGGRSRTSIILTVGPSSDHFYETTNSLQFGLRAMAVKVSAKQSVVVNYEKLAHNLQMLLDEKQERIALLEIQIAGRDAERAELMERYNKHRAEIDLRYERDMARLMASNAPPEKIEGLREVYRVEVENLHEQRDEEIQYQEEAHSKEITKLVREQAEQEAKRRAEMKLAQERIIEDFQKKLDNAREGKHDDIVNALRQLAEKDSLLASRANDTARLHEHIEVLTQQIREMGGTPVEEAVFPETFLDVGQVEEIQQRLEAEVERHREKEVQLYAEVERLSRICSERVEEINKLHDENEQLRSDLSNKGFNVGETDELTKYLRDRRAKMIDSSEMETLRVTMRAEMEELKAHNVELKREVERLEEERAQQSLPLTARIFGTARNSLSTARSVLPAGGAPPLTGRGASQLRQYFLSDDTRSLSHSFNGETSKKAVKELSDQLTFSIQEKNALLNRIQKLEAELKSHGVESPQPYVPPIMLGSSVLPNIPSLVAPSPDAAPNADVDVLLRVKDAEVDEFMETIERQQHLLATARSNDEHYQQVISDLRQAIAKAGLPLPESQAIPSPVDCIAMDDYMNILRAVRDSERKMVVRLAEREGKDPLEIDSVLEEMNRELIFKDELVIENASKMQFVAKVCIRLKSQLERLGITPCCQLPDSYKELIEREKCEMEEQTEAQRDLEEKLSMLAEEKQRLNKMLSSMRQEREMDIVVMRSVQERCKEAEEKEIYAAEALSRLTREKSQKERALEETLRLATMDLMQYQAQLAQIKELENTGGFARILKLLLRR

(19) United States
(12) Patent Application Publication
(10) Pub. No.: US 2018/0185461 A1
(43) Pub. Date: Jul. 5, 2018

(54) TREATMENT AND DETECTION OF TRYPANOSOMES

(71) Applicants: UNIVERSITE DE BORDEAUX, BORDEAUX (FR); CENTRE HOSPITALIER UNIVERSITAIRE DE BORDEAUX, TALENCE CEDEX (FR); UNIVERSITE LIBRE DE BRUXELLES, GOSSELIES (BE); INSTITUT DE RECHERCHE POUR LE DEVELOPPEMENT, MARSEILLE CEDEX 02 (FR)

(72) Inventors: PHILIPPE VINCENDEAU, PESSAC (FR); JEAN-LOUP LEMESRE, MONTPELLIER (FR); ETIENNE PAYS, NIL SAINT VINCENT (BE)

(21) Appl. No.: 15/575,047

(22) PCT Filed: May 18, 2016
(86) PCT No.: PCT/FR2016/051170

§ 371 Date: Nov. 17, 2017

(30) Foreign Application Priority Data
May 19, 2015 (FR) 1554450

(51) Int. Cl. A61K 39/005; A61P 33/02; C07K 16/20; G01N 33/569

(52) U.S. Cl. CPC A61K 39/005; A61P 33/02; C07K 16/20; G01N 2800/52; C07K 2317/76; G01N 2800/26; G01N 33/56905

(57) ABSTRACT

The present invention relates to methods and compositions for preventing, treating and diagnosing infection by trypanosomes. The invention also relates to the use of excreted/secreted antigens (exoantigens, secretome) and specifically to the identification of a protein excreted/secreted by the trypanosomes, the inhibition of which makes it possible to provide effective protection, mainly by vaccination, against infection by trypanosomes or the development or spread thereof. The invention relates to use of the protein, the derivatives thereof, a nucleotide sequence derived from said protein, or an extract enriched with said protein, and to the use of antibodies directed against said trypanosomes for immunotherapy, diagnosis, and monitoring of infections by trypanosomes.

The present invention relates to methods and compositions for preventing, treating and diagnosing trypanosome infection. In particular, it relates to the use of excreted/secreted antigens (exoantigens, secretome) and, more particularly, to the identification of a protein excreted/secreted by trypanosomes, the neutralization or inhibition of which makes it possible to confer effective protection against infection with trypanosomes or the development or spread thereof, mainly by vaccination. The invention enables a cross-action against different strains of trypanosomes, and thus provides effective methods and compositions for preventing and controlling infections and pathologies induced by trypanosomes in mammals, for more precisely diagnosing same, and for following the evolution of the infection after treatment.

### INTRODUCTION

Trypanosomes (Trypanosoma) are parasitic protozoa infecting mainly mammalian animals, but also humans. In animals, infection causes trypanosomiasis (sometimes called trypanosomosis), which can cause the animal to die. In man, human African trypanosomiasis begins with an inoculation canker, followed by a hemolymphatic stage with, in particular, fever, adenopathy, hepatosplenomegaly, pruritus and edema. A meningoencephalitic stage follows, when the parasite enters the central nervous system, with various neurological signs and, in particular, sleep disturbances (whence the name “sleeping sickness”). Chagas disease (human American trypanosomiasis) is caused by Trypanosoma cruzi, transmitted by bugs. When T. cruzi penetrates the skin, an erysipeloid or pseudo-furuncular skin lesion (chagoma) may appear, then sometimes unilateral bi-palpebral edema (Romaña's sign). The acute phase may pass unnoticed, and more rarely may manifest as febrile hepatosplenomegaly. The chronic phase is dominated by the gravity of cardiac forms and the existence of digestive forms with mega-organs.

Trypanosomes are characterized by high genetic diversity, which influences tropism, virulence, transmissibility and sensitivity to trypanocides. Among the various groups of trypanosomes, particular mention may be made of the Stercoraria group, which includes Trypanosoma cruzi, T. theileri, T. lewisi and T. musculi, and the Salivaria group, which includes three main subgenera: Trypanozoon, Duttonella and Nannomonas. The subgenus Trypanozoon comprises species of trypanosomes with extracellular development that infect animals and humans, whereas Duttonella and Nannomonas infect only non-human mammals. The subgenus Trypanozoon consists of polymorphic trypanosomes (long form and short or squat form), with an optional free flagellum and a small kinetoplast in the subterminal (posterior) position. The main species of this subgenus are Trypanosoma (T.) brucei, T. evansi and T. equiperdum. T brucei includes three subspecies: T. b. brucei, T. b. gambiense and T. b. rhodesiense, which are quite similar in morphological, antigenic and biochemical terms, and which are distinguished by their infectious nature, pathogenicity and geographical distribution. T. brucei and subspecies thereof are transmitted by tsetse flies (Glossina). T. evansi is transmitted to cattle, horses and camels by biting flies other than tsetse (Tabanidae) in Africa, South America and Southeast Asia. T. equiperdum has no invertebrate host (sexual transmission in horses), and has been detected in Europe, Asia, Africa and America. Trypanosomes of the subgenus Duttonella are club-shaped. The main species are T. vivax and T. uniforme, which have a tropism for wild and domestic ruminants. Trypanosomes of the subgenus Nannomonas are small and have no free flagellum. The main species are T. congolense and T. simiae, which have a strong tropism for cattle, pigs and dogs.

In Africa, T. congolense, T. vivax, T. brucei and T. evansi are the principal agents responsible for trypanosomiasis, notably in domestic mammals such as ruminants, cattle, pigs, sheep, goats, horses and dogs. T. brucei, and notably the subspecies T. b. gambiense, is probably the most well-known since it is responsible for the chronic form of sleeping sickness in humans in Western and Central Africa. The subspecies T. b. rhodesiense is the agent responsible for the acute form of sleeping sickness. T. vivax is a parasite mainly of ungulates in tropical Africa and is transmitted by horseflies (Tabanidae). T. equiperdum is also present in Africa. The subspecies T. evansi is transmitted to cattle, horses and dromedaries, and has significant economic repercussions throughout the cattle-rearing regions. Human cases caused by T. evansi are exceptional. Rare cases of trypanosomiasis caused by other species of trypanosomes (trypanosomes of the lewisi, T. theileri group) have been reported in humans and in animals.

Trypanosomes have a complex life cycle that includes various morphological forms, depending on the subspecies. Generally, during infection, the tsetse fly (Glossina) injects into the host's dermis at the puncture site the infectious metacyclic forms. The parasites multiply in the dermis at the inoculation point, giving rise to blood forms. This stage can last from 1 to 3 weeks. The parasites then invade the blood, the lymphatic system, and various organs such as the heart or the kidneys, where they cause significant lesions. The sources of infection for domestic animals are also other infected domestic animals or wild animals that are sick or are healthy carriers.

At present, control of the disease involves mainly control of the vectors by means of insecticides, used in particular to impregnate traps, which has an environmental impact. In South America, control of the bugs that are vectors of Chagas disease involves persistent insecticides sprayed within dwellings, associated with improved living conditions. In infected mammals, the ability of trypanosomes to escape the host's immune defenses by expressing variable antigens on their surface has to date prevented the development of effective vaccine strategies. Only a few trypanocidal molecules are available, but they cause significant side effects and many resistant parasite strains have appeared. In diagnostic terms, diagnosis is generally limited to a suspicion based on observation of symptoms. But there are to date no reliable markers allowing rapid and specific detection of infection, at a reasonable cost.

There is thus a need in the prior art for effective approaches to preventing, treating and detecting trypanosome infections.

### SUMMARY OF THE INVENTION

The present invention relates to methods and compositions for treating and diagnosing trypanosome infection. It relates to the use of excreted/secreted antigens (exoantigens, secretome) and, more particularly, to the identification of a protein secreted by trypanosomes, the neutralization (by antibodies acquired by vaccination or injection) or inhibition (by various molecules) of which confers an effective protection against infection with trypanosomes or the development or spread thereof. The invention enables a cross-action against different strains of trypanosomes, and thus provides effective methods and compositions for controlling infections and pathologies induced by trypanosomes in their mammalian hosts. It also makes it possible to detect said protein and antibodies against same in any sample, and to monitor the evolution of the trypanosomiasis, with or without treatment. It also allows the construction of primers and probes that enable the use of various molecular biology techniques, such as the polymerase chain reaction (PCR) applied to the diagnosis of trypanosomiasis.

An object of the invention thus concerns pharmaceutical or veterinary compositions comprising (i) TbKHC1 protein or one or more antigenic peptides thereof, a nucleic acid encoding said protein or said peptide, or an inhibitor of TbKHC1 protein and (ii) a pharmaceutically or veterinarily acceptable excipient.

In a particular embodiment, the invention relates to compositions, such as vaccines, comprising (i) TbKHC1 protein or one or more antigenic peptides thereof, or a nucleic acid encoding said protein or said peptide, (ii) a pharmaceutically or veterinarily acceptable excipient, and (iii) optionally an adjuvant selected advantageously to strengthen an immune response.

In a particular embodiment, the invention relates to compositions, such as vaccines, comprising (i) TbKHC1 protein, or one or more antigenic peptides thereof, complexed to or in association with one or more other trypanosome molecules, (ii) a pharmaceutically or veterinarily acceptable excipient, and (iii) optionally an adjuvant selected advantageously to strengthen an antibody response.

In another particular embodiment, the invention relates to compositions comprising (i) an anti-TbKHC1 antibody, or a fragment or derivative of such an antibody, and (ii) a pharmaceutically or veterinarily acceptable excipient.

The invention also has as an object a composition as defined above, or TbKHC1 protein or one or more antigenic peptides thereof, or a nucleic acid encoding said protein or said peptide, for use to vaccinate or immunize a mammal against trypanosomes and/or trypanosomiasis.

The invention also has as an object a composition as defined above, or TbKHC1 protein or one or more antigenic peptides thereof, or a nucleic acid encoding said protein or said peptide, for use to protect a mammal against trypanosomiasis.

The invention also has as an object a composition as defined above, or TbKHC1 protein or one or more antigenic peptides thereof, or a nucleic acid encoding said protein or said peptide, or an inhibitor thereof, for use to treat a mammal with trypanosomiasis.

The invention further relates to the use of TbKHC1 protein or one or more antigenic peptides thereof, or a nucleic acid encoding said protein or said peptide, or a secretion extract enriched in said protein, for the preparation of a vaccine to immunize or protect a mammal against trypanosomes.

According to another aspect, the invention relates to the use of an inhibitor of TbKHC1 protein for the preparation of a medicinal product for treating a mammal with trypanosomiasis.

The invention also relates to a method for treating a mammal with trypanosomiasis, comprising inhibiting TbKHC1 protein in said mammal. Inhibition may be obtained by administering an inhibitor (for example an antibody or a molecule interfering with the binding or the function of said protein in mammalian host tissue), or by vaccinating said mammal against TbKHC1 protein or an antigen thereof. The invention thus proposes novel immunotherapies for trypanosomiasis using any, notably monoclonal, anti-TbKHC1 antibody or derivatives of such antibodies or constructions using the amino acid or nucleotide sequence of a portion of such antibodies.

The invention also has as an object any antibody specifically binding to TbKHC1 protein.

Another object of the invention relates to a method for in vitro diagnosis of trypanosomiasis in a mammal, characterized in that it comprises identifying and/or measuring, in a sample from said mammal, the presence of TbKHC1 protein or antigenic peptides thereof or antibodies against said protein.

Another object of the invention relates to a method for monitoring the evolution of trypanosome infection in a mammal, characterized in that it comprises identifying and/or measuring the amount of TbKHC1 protein or antibodies against said protein in samples from the mammal taken at various time intervals.

Another object of the invention relates to a method for determining the efficacy of a treatment against trypanosomes in a mammal, characterized in that it comprises identifying and/or measuring the amount of TbKHC1 protein in samples from the mammal or of antibodies against said protein taken at various time intervals during the treatment, and optionally after the treatment.

The invention further relates to:
kits for measuring trypanosomes in a test sample, characterized in that said kits comprise at least one antibody as defined above, a medium suitable for the formation of an immune complex with said antibody, and at least one reagent for detecting an immunological reaction;
kits for detecting antibodies against TbKHC1 protein for diagnosing infection using TbKHC1 protein, peptides, or natural or synthetic epitopes derived from said protein.

Another object of the invention relates to any diagnostic method using the nucleotide sequence of TbKHC1 protein for diagnosing trypanosomiasis or for precisely identifying a species of trypanosomes (for example, the construction of primers or probes enabling the use of various molecular biology techniques such as PCR or hybridization).

The invention may be used to prevent, treat, detect or monitor the evolution of after vaccination or treatment any disease caused by parasites of the genus Trypanosoma, in any mammal, notably in domestic or livestock animals, and in humans.

### BRIEF DESCRIPTION OF THE FIGURES

FIG. 1: A TbKHC1 inhibitor inhibits parasite proliferation in vitro.

FIG. 2: A TbKHC1 inhibitor reduces parasite load in vivo.

FIG. 3: Vaccination strategy.

FIG. 4: Survival rate of mice (protective effect) with parasitic infection after vaccination according to the invention.

FIG. 5: Detecting infection by measuring antibodies against TbKHC1 protein.

FIG. 6: Seroprotection test: Naive mice receive, 24 hours before infection by T. Feo (2000 parasites), 300 μL of serum from mice immunized with the total secretome of T. b. gambiense Feo (“Serum total PSF”) or the fraction containing high molecular weights greater than 100 kDa (“Serum HMW>100”) or the fraction containing high molecular weights greater than 50 kDa (“Serum HMW>50”) or the fraction containing low molecular weights lower than 50 kDa (“Serum LMW<50”) or the fraction containing molecular weights between 50 and 100 kDa (“Serum 100<MW>50”). Mouse survival is measured according to the number of days post-infection by T. Feo.

FIG. 7: Cross-seroprotection test. Mice receive, 24 hours before infection by T. b. brucei (2000 parasites), 300 μL of serum from mice immunized with the total secretome of T. b. gambiense Feo (“Serum total PSF”) or the fraction containing high molecular weights greater than 50 kDa (“Serum HMW>50”) or the fraction containing low molecular weights lower than 50 kDa (“Serum LMW<50”) or the serum of naive mice. Mouse survival is measured according to the number of days post-infection by T. b. brucei.

FIG. 8: A: Mouse rate of survival to parasitic infection after vaccination according to the invention: mice are immunized twice, in the presence of adjuvant (saponin), with either the total secretome of T. b. gambiense Feo (“Total PSF”) or the fraction containing high molecular weights greater than 50 kDa (“HMW>50”) or the fraction containing low molecular weights lower than 50 kDa (“LMW<50”). Control mice receive adjuvant alone (“Controls”). The mice are infected 2 months thereafter with T. b. brucei (2000 parasites). Mouse survival is measured according to the number of days post-infection by T. b. brucei. B: Mouse rate of survival to parasitic infection after vaccination according to the invention: mice are immunized twice, in the presence of adjuvant (saponin), with the total secretome (“Total PSF”) of T. b. brucei or T. b. brucei KO for kinesin. Control mice receive adjuvant alone (“Controls”). The mice are infected 2 months thereafter with T. b. brucei (2000 parasites). Mouse survival is measured according to the number of days post-infection by T. b. brucei. C: Mouse rate of survival to parasitic infection after vaccination according to the invention: mice are immunized twice, in the presence of adjuvant (saponin), with the total secretome of T. evansi (“Total PSF”). Control mice receive adjuvant alone (“Controls”). The mice are infected 2 months thereafter with T. b. brucei (2000 parasites). Mouse survival is measured according to the number of days post-infection by T. b. brucei.

FIG. 9: Protein profile obtained by electrophoretic migration of 5 μg of each sample under denaturing and non-reducing conditions and then Coomassie blue staining. The boxed region identifies the protein bands that may contain TbKHC1.

FIG. 10: Immunoblot of 1 μg protein equivalent of antigen after semi-dry transfer (3.5 h; 24 mA). Primary antibody (purified Mab1 antibody) diluted 1:200; secondary antibody (total mouse anti-IgG) diluted 1:5000.

FIG. 11: Immunoblot set-up. Deposition of 1 μg of antigen, wet transfer (O/N; 4° C.; 10 mA). Primary antibody (serum anti-PSF T. Feo) diluted 1:200; secondary antibody (total mouse anti-IgG) diluted 1:5000.

FIG. 12: Immunoblot set-up. Deposition of 1 μg of antigen, wet transfer (O/N; 4° C.; 10 mA). Primary antibody (serum anti-HMW50 T. Feo) diluted 1:200; secondary antibody (total mouse anti-IgG) diluted 1:5000.

### DETAILED DESCRIPTION OF THE INVENTION

The present invention relates to methods and compositions for preventing, treating, diagnosing and monitoring the evolution of trypanosome infection based on neutralizing or inhibiting TbKHC1 protein and on detecting same or antibodies against same. The invention makes it possible to confer effective protection against infection with different strains of trypanosomes and the development or spread thereof, mainly by vaccination. It can be used in any mammal.

### Definitions

The term “trypanosomiasis” or “trypanosomosis” refers, in a general way, to all disorders caused by a trypanosome in mammals. The term “trypanosomiasis” notably includes nagana, surra, dourine, sleeping sickness, African trypanosomiasis, American trypanosomiasis, Chagas disease, and all lesions caused to organs (e.g., kidney, heart, liver, testicle, digestive tract, brain) by trypanosome infection.

The term “treatment” or “to treat” refers to any improvement in the subject's condition. The treatment may be curative or preventive. Curative treatment is intended for an infected mammal and aims to stop, reduce, slow or delay the development of disease in the infected mammal. It notably includes, in an infected subject, reduction of parasitic load, disappearance of the parasite, reduction of proliferation or transmission of same, reduction of disorders caused by the parasite and notably lesions to organs, reduction of symptoms, or total eradication of the disease. Curative treatment typically uses an inhibitor of the pathogen (immunotherapy or chemotherapy). Preventive treatment is intended for a mammal not infected with the parasite and aims to stop, prevent or reduce infection in a healthy mammal. Preventive treatment generally uses an antigen of the pathogen, to generate a protective immune response.

### Identification of a Virulence Factor

The invention follows from the identification of TbKHC1 protein, secreted by trypanosomes, the neutralization or blocking of which inhibits the proliferation of the parasite and the transmission and virulence of same. The invention further shows that immunization with a preparation containing TbKHC1 protein produced by different trypanosomes is possible and induces cross-protection against different types of trypanosomes. Said protein thus represents a particularly relevant and attractive target for any therapeutic or diagnostic strategy against trypanosomes and trypanosomiasis.

An object of the invention thus concerns TbKHC1 protein, or one or more antigenic peptides thereof, or a nucleic acid encoding said protein or said peptide, or an inhibitor of TbKHC1 protein, for use in the preventive or curative treatment of trypanosome infection in a mammal. The invention also concerns the use of TbKHC1 protein, or one or more antigenic peptides thereof, or an inhibitor thereof, to treat trypanosome infection in a mammal. The invention also relates to a method for treating trypanosome infection in a mammal comprising inhibiting (e.g., reducing, neutralizing or blocking) TbKHC1 protein in the mammal. Inhibiting the protein comprises reducing the amount of or inhibiting the activity of the protein and may be obtained for example (i) by immunizing or vaccinating the mammal with a preparation containing TbKHC1 protein, for example by administering an immunogenic amount of TbKHC1 protein or one or more antigenic fragments thereof; and/or (ii) by inhibiting TbKHC1 protein present in the mammal, by administering an inhibitor or a competitor thereof.

Thus, within the meaning of the invention, the term “to inhibit” or “inhibition of” a protein refers to any reduction of the amount or the activity of said protein. Inhibition thus notably refers to lowering or reducing the amount of said protein by compounds affecting the synthesis, secretion or structure thereof. Inhibition also refers to any decrease in the activity of the protein by compounds (antibodies or derivatives, peptides, chemical molecules) acting directly thereon or on one or more target molecules thereof in mammalian hosts, in order to interfere with the action of said protein.

Within the meaning of the invention, the term “TbKHC1 protein” refers to a protein comprising the amino acid sequence represented in SEQ ID NO: 2 or any natural variant of said sequence resulting from polymorphisms or variations between species or subgroups of trypanosomes, or any kinesin-type protein secreted by a trypanosome and having a sequence with at least 45% sequence identity with SEQ ID NO: 2, preferably at least 60%, more preferentially at least 70%. Even more preferentially, the sequence identity with SEQ ID NO: 2 is 80% or higher, preferably at least 85%, 90%, 95%, 96%, 97%, 98%, 99% or higher. Sequence SEQ ID NO: 2 is represented below. It corresponds to TbKHC1 protein of T. brucei brucei:

MSDADVKEGT AAGDSVAVPE SVVKPDEGRR SRGESTGGTA
AGDTGVPKNI ARCLVYCRLR PRNKTDFKNG GFQLVTVSGN
DIVVKDQRFY KFDGAFGDEC TQSDIFEAVA VPCITHAFKG
FCSALMCYGQ TGTGKSFTMC NTTPGQEGII PRSAKLIFDK
IQSDNARSYE VTGQFVQIYR DNLGDLMSAT GRDRVDIHFD
EQGGVELTGC SSHVLLSAQE FMRFYRIGND RRVVTATAMN
PESSRGHTAL VLRIVSESPS DPEAGKLKGK ITFIDLAGYE
RFSKTGITHD NPIMKDEAKC INASLLSLGH VVSCLSSGSR
HIPWRDSKLT RILQDSIGGR SRTSIILTVG PSSDHLHETT
NSLQFGLRAM DVKVTAKQSV HVDYQKLAQK LQSLLDERDE
RINLLEVQIA SRDAERHELM ERYNDRREDI DRRFEIEMAE
LKRTGASEEQ MLNLREVYKA EVENLQEQQD EEFQYREEVY
SKEIVHLIRE QEHQEAKRRA EMKLAQDLII AEFQKKLDNA
REGTNDDLVR VLKQLSEKDA ILASRANDTV RLHEHIEVLR
EQVKELGGVP IEEATFPETF LDVGQVEEMR NRLEADVQRH
RAKGVELLAE VDRLSQLCSE RLEEINRLRD ENTQYRAALE
NSGISLNDTD DLTEFLSEKR TQMVDVSEME TLRVTMQADL
DEAKAHNREL AREVEQLKFE LTATAIPLTA RLRCPPCATA
RGPSPFDAAR NLCSTQRKPP QKDGTPSPNN TQNENLQRTV
KQLTEQLEFS MRERKSLQDR VEAVETQLAS HGVEVPGPYV
PPIKLGFPGS APVTSSETDA REPPEDTDMD VLLRVKEEEI
DVLLETIERQ EHLLNAARSN EEFHRRVICE LQQQMVTAQI
QVEDPQNAPP PVDAIAMDEY MSILRLVRES ERKLAAQLAE
RDGEDGAEVE ALLEKKDAEL QMKEETILEK ASKAQYAAKL
CIRLKNQMLR CGITPCCELP DSYNELIERE EEELNEQLMC
QDELLARLRS EEEEKHRMQN MLKSLNEERE RQSSVIRTVQ
ERCELVEKKQ LVTAAHLSRL ATEKSQREQI LEETLRRATQ
ELLDCKIKMA MEKEAGSPGV LKRFLRRLRS N

Research carried out by the Inventors identified other TbKHC1 proteins within the meaning of the invention from other species of trypanosomes, notably from T. brucei gambiense (99% identity with SEQ ID NO: 2); T. brucei rhodesiense; T. evansi; T. equiperdum; T. congolense (76% identity with SEQ ID NO: 2); T. vivax (69% identity with SEQ ID NO: 2); T. musculi (a parasite of the lewisi group (61% identity with SEQ ID NO: 2), and T. cruzi (61% identity with SEQ ID NO: 2). The sequence of TbKHC1 proteins of these species is represented in SEQ ID NO: 3 (Trypanosoma brucei gambiense), SEQ ID NO: 4 (Trypanosoma congolense), SEQ ID NO: 5 (Trypanosoma vivax), and SEQ ID NO: 6 (Trypanosoma cruzi). These proteins represent examples of TbKHC1 proteins within the meaning of the invention. Furthermore, persons skilled in the art may, on the basis of the information provided in the present application and on conventional techniques, identify other TbKHC1 from other subgroups of trypanosomes. In this context, the term “sequence identity,” applied to a nucleic acid or a protein, refers to the quantification (generally expressed as a percentage) of the matching of nucleotide or amino acid residues between two aligned sequences using a standard algorithm such as Smith-Waterman alignment (Smith and Waterman (1981) J Mol Biol 147:195-197), CLUSTALW (Thompson et al. (1994) Nucleic Acids Res 22:4673-4680; Altschul et al. (1997) Nucleic Acids Res 17:3389-402), or BLAST2 (Nucleic Acids Res 25:3389-3402). BLAST2 may be used in a standardized and reproducible manner to insert gaps in one of the sequences in order to optimize the alignment and to achieve a more significant comparison.

TbKHC1 protein may be obtained in different ways. It may be in pure, enriched extract (for example enriched secretion extract), recombinant or synthetic form, etc. It may first be isolated in eluted fraction form or purified from a culture of trypanosomes. To that end, purified parasites are preferentially incubated in secretion medium (for example of type Ringer lactate+glucose), then the secretory products (secretome) are collected, for example by centrifugation, filtered to sterilize, then passed through an affinity column comprising anti-TbKHC1 antibody. After washing, the molecules retained on the column are eluted, producing an extract or a fraction comprising TbKHC1 protein and/or fragments thereof. In a variant, TbKHC1 protein is obtained from the secretome by differential filtration on filters having cut-offs of 50 or 100 kDa, making it possible to separate a fraction containing molecules of high molecular weights (HMW) and a fraction containing molecules of low molecular weights (LMW). TbKHC1 protein is present in the HMW fractions, in particular HMW50 and HMW100.

The fraction enriched in TbKHC1 protein obtained may further comprise proteins or peptides of different nature from the trypanosome. In particular, as TbKHC1 protein has the property of binding to and/or transporting other molecules of the trypanosome notably by virtue of its coiled coil structure, which promotes interaction with other molecules, the fraction obtained may comprise TbKHC1 protein, or peptides thereof, complexed or associated with other trypanosome proteins or peptides or molecules. Moreover, TbKHC1 protein may be further concentrated and/or purified from said fraction, in order to obtain a purity greater than 90%, for example of 95% or more, notably of 98% or more, in particular a TbKHC1 protein free of any other trypanosome protein.

In this respect, the invention also aims at a method for preparing an antigenic fraction, comprising obtaining a trypanosome secretome, differential filtration of the secretome on a filter having a 50 or 100 kDa cut-off, and collecting the high molecular weight fraction. This process has various advantages: it makes it possible to improve the production yield of TbKHC1-enriched fractions, it improves reproducibility (similar protein assay for the various batches), and it preserves the immunogenicity of the antigens (differential filtration is less detrimental to the antigenic structure than acid elution). The invention also relates to the preparation obtained by this process and the veterinary or pharmaceutical use thereof, as illustrated in the present application.

TbKHC1 protein may also be produced recombinantly, by expressing in a host cell an encoding nucleic acid. In this respect, another object of the invention concerns a process for producing TbKHC1 protein, comprising culturing a recombinant cell comprising a nucleic acid encoding TbKHC1 under conditions allowing the expression and, optionally, the secretion of TbKHC1 protein and then collecting and, optionally, purifying TbKHC1 protein. The cell used may be prokaryotic (for example a bacterium such as E. coli) or eukaryotic (for example a yeast, a mammalian cell or an insect cell). The nucleic acid encoding TbKHC1 may be DNA or RNA, and the sequence thereof may be determined by persons skilled in the art according to the protein sequence to be encoded. By way of illustration of a sequence encoding the protein of SEQ ID NO: 2, mention may be made of nucleotide sequence SEQ ID NO: 1, which is represented below:

ATGTCGGATG CCGATGTGAA AGAGGGAACG GCGGCCGGCG
ATTCAGTGGC CGTTCCCGAG TCGGTTGTAA AACCAGATGA
AGGACGGCGG AGCAGAGGTG AGTCTACTGG CGGGACAGCT
GCTGGGGATA CCGGTGTGCC AAAGAATATA GCACGGTGTC
TTGTTTATTG CAGGTTGAGG CCACGGAACA AGACTGATTT
TAAGAACGGT GGGTTCCAAC TAGTGACAGT AAGCGGGAAT
GATATTGTTG TGAAGGATCA ACGCTTTTAC AAGTTTGATG
GTGCTTTTGG CGACGAATGT ACACAAAGTG ATATATTTGA
AGCGGTGGCC GTCCCTTGCA TAACACACGC ATTTAAAGGT
TTTTGCTCAG CGTTGATGTG CTACGGACAG ACGGGTACAG
GTAAGTCTTT CACTATGTGT AATACCACTC CTGGCCAAGA
AGGCATCATT CCACGGTCCG CCAAACTTAT TTTCGACAAA
ATTCAATCAG ACAATGCGCG GAGTTATGAA GTGACAGGAC
AGTTTGTTCA GATTTACCGT GACAACCTTG GTGACTTGAT
GAGTGCAACT GGAAGGGACC GAGTGGATAT TCACTTCGAC
GAACAAGGGG GCGTAGAACT TACCGGTTGC AGCTCCCATG
TTCTTCTGAG TGCCCAAGAG TTTATGCGCT TTTACCGCAT
CGGCAATGAC CGTCGGGTTG TAACTGCGAC TGCTATGAAT
CCGGAGTCCA GCCGCGGCCA TACAGCTTTA GTTCTCCGCA
TCGTATCAGA GAGCCCCAGC GACCCAGAGG CAGGTAAACT
GAAGGGAAAG ATTACATTCA TCGACTTAGC AGGATACGAG
CGTTTTAGTA AAACTGGTAT TACACATGAC AACCCCATTA
TGAAGGATGA GGCGAAGTGC ATCAACGCCT CTCTTCTTTC
ACTTGGTCAC GTTGTGTCGT GTTTGTCGTC AGGTAGCCGG
CACATTCCTT GGCGTGATTC GAAGCTGACG CGGATCCTGC
AGGACTCTAT TGGCGGAAGG AGCCGTACCT CTATTATTTT
GACTGTTGGG CCAAGTAGTG ATCACCTCCA CGAAACCACA
AATTCACTGC AGTTTGGTTT GCGAGCAATG GATGTGAAGG
TGACGGCCAA ACAGTCGGTT CATGTGGATT ACCAGAAGCT
GGCCCAGAAG CTGCAATCAC TCTTGGATGA AAGGGACGAG
AGAATCAATT TACTCGAAGT GCAGATCGCT TCTCGTGACG
CAGAAAGACA CGAGTTAATG GAGCGTTACA ACGATCGCCG
GGAAGACATT GACAGACGTT TTGAGATTGA GATGGCTGAA
CTGAAGAGAA CTGGTGCATC GGAAGAGCAG ATGCTGAACC
TGCGTGAAGT ATACAAGGCT GAGGTGGAAA ACCTCCAGGA
GCAGCAAGAC GAGGAGTTCC AATACAGGGA GGAAGTGTAT
TCAAAGGAGA TCGTCCACCT TATTCGCGAG CAGGAGCATC
AGGAAGCGAA GCGACGGGCA GAGATGAAAT TGGCGCAAGA
TCTTATCATT GCGGAGTTCC AAAAGAAGCT CGACAACGCG
CGTGAGGGAA CAAATGATGA TCTCGTCAGA GTTTTGAAGC
AACTGTCCGA AAAGGACGCC ATATTGGCCA GCCGAGCGAA
CGACACGGTG AGACTCCACG AACATATTGA GGTGCTCAGG
GAGCAAGTGA AGGAGCTCGG TGGAGTGCCT ATAGAGGAGG
CGACGTTTCC CGAAACCTTT CTGGACGTTG GCCAGGTGGA
GGAGATGCGG AACCGGCTGG AGGCGGATGT GCAACGCCAT
CGTGCTAAGG GTGTGGAATT GCTTGCGGAA GTGGATCGTC
TTTCGCAGCT CTGCTCTGAG CGGTTGGAGG AGATAAACCG
ACTCCGCGAC GAAAACACAC AATATCGCGC CGCATTGGAA
AACAGTGGCA TTTCATTGAA TGACACTGAT GATTTGACGG
AATTCCTTTC TGAGAAGCGC ACTCAGATGG TGGATGTTTC
TGAGATGGAA ACTCTTCGTG TCACCATGCA GGCCGACCTT
GATGAAGCGA AGGCGCACAA CCGGGAGCTG GCGCGGGAGG
TGGAGCAGTT GAAGTTTGAA TTAACCGCAA CCGCTATTCC
ACTCACAGCC CGGCTTCGAT GTCCGCCGTG CGCAACTGCA
CGAGGTCCTT CCCCGTTTGA CGCCGCGCGC AACCTGTGTT
CGACGCAGCG TAAACCACCT CAAAAGGATG GCACGCCATC
CCCAAACAAC ACTCAAAATG AAAACTTGCA AAGGACCGTG
AAGCAGCTTA CGGAGCAACT GGAATTCAGC ATGCGTGAGA
GGAAGTCGCT TCAGGACCGC GTTGAGGCTG TTGAGACGCA
ACTTGCTTCG CATGGTGTTG AGGTTCCGGG GCCGTACGTA
CCCCCAATCA AACTTGGTTT CCCCGGCTCT GCACCAGTGA
CGTCATCGGA AACAGATGCA AGGGAGCCAC CGGAGGATAC
CGATATGGAT GTGCTGCTCC GTGTAAAAGA GGAGGAAATC
GATGTGTTAT TGGAAACAAT TGAACGGCAG GAGCACTTGC
TCAATGCTGC GAGGTCGAAT GAAGAGTTTC ACCGACGCGT
CATTTGTGAG TTGCAGCAGC AGATGGTGAC TGCGCAAATC
CAGGTGGAAG ATCCTCAGAA CGCCCCTCCT CCTGTTGACG
CCATTGCAAT GGATGAGTAT ATGTCAATTT TGCGTTTAGT
TCGGGAGTCC GAACGCAAGT TGGCAGCTCA ATTGGCTGAG
CGCGATGGAG AGGATGGCGC GGAGGTGGAG GCCCTGTTGG
AGAAGAAGGA TGCGGAACTA CAAATGAAGG AGGAGACCAT
ACTCGAGAAG GCGTCGAAGG CGCAGTATGC AGCGAAGCTC
TGCATTCGTC TGAAGAACCA GATGCTGCGT TGTGGCATCA
CACCGTGTTG TGAGCTTCCA GACTCGTATA ACGAGTTGAT
CGAGCGCGAA GAGGAGGAAC TGAATGAGCA ACTAATGTGC
CAAGATGAAC TGTTAGCCAG GCTTCGTTCG GAGGAGGAAG
AAAAGCATCG CATGCAGAAT ATGCTGAAAT CACTTAATGA
GGAGCGCGAG AGGCAATCCA GCGTCATTCG AACTGTTCAA
GAGCGCTGTG AACTGGTGGA AAAGAAACAA TTGGTTACGG
CAGCCCACTT GTCGCGATTG GCAACGGAAA AATCCCAGAG
GGAGCAAATT CTTGAGGAAA CGCTACGACG TGCAACACAA
GAATTGTTGG ATTGCAAGAT TAAGATGGCC ATGGAAAAAG
AAGCAGGTAG CCCGGGTGTG TTAAAGCGTT TCCTCCGCCG
CCTGCGCTCC AACTGA

The nucleic sequence may further be optimized for expression in the selected host cell. Another object of the invention concerns an expression vector comprising a nucleic acid encoding TbKHC1 protein, preferably under the control of a promoter. Another object of the invention concerns a host cell containing such a vector, or containing a nucleic acid encoding TbKHC1 protein inserted in the genome thereof.

TbKHC1 protein may also be obtained by artificial synthesis, using protein synthesizers. It may also be produced by a combination of said methods.

Within the meaning of the invention, the term “antigenic peptide” or “antigenic fragment” refers to a peptide the sequence or a portion of the sequence of which corresponds to a portion of the sequence of TbKHC1 protein, and which is capable of inducing an immune response against TbKHC1 protein. An antigenic peptide thus generally comprises at least one specific epitope of TbKHC1 protein, making it possible to induce an immune response specifically against TbKHC1. The term “peptide” refers, within the meaning of the invention, to a molecule having from 4 to 500 amino acids, for example from 4 to 450 amino acids, for example from 4 to 300 amino acids, or fewer, for instance from 4 to 50, 40 or 30, or even fewer. Examples of antigenic peptides within the meaning of the invention include peptides comprising at least residues 1000-1111, 900-1111, 800-1111, 700-1111, 687-1111 or 500-1111 of sequence SEQ ID NO: 2 or natural variants thereof. A particular antigenic peptide is notably a peptide comprising residues 687-1111 of SEQ ID NO: 2.

The protein or the antigenic peptides according to the invention may comprise modifications, notably chemical modifications, that do not alter their immunological specificity. Thus, notably, they may be chemically modified to improve their stability, their tropism, their solubility or their immunogenicity. Examples of modifications include the addition of phosphates, sugars or myristic acids, or polyethylene glycol. In the more particular case of peptides, they may comprise, in addition to the immunogenic sequence, one or more residues promoting expression, stability or immunogenicity. Peptides may also be coupled to carrier molecules, or to other epitopes, in order to increase their immunological potential, or complexed or associated with other proteins to increase their immunogenicity. Particular peptides of the invention are peptides consisting of an immunogenic sequence of TbKHC1 protein.

### TbKHC1 Inhibitor

The invention also relates to any TbKHC1 inhibitor and to the use thereof for treating trypanosome infections. The term “TbKHC1 inhibitor” refers to any compound capable of reducing the amount (for example the production or the secretion) or the activity of TbKHC1 protein. It is typically a specific inhibitor, i.e., one capable of acting on TbKHC1 with no direct effect on other proteins produced by the trypanosome or by the infected mammal. The inhibitory compound may be a ligand of TbKHC1 protein, for instance an antibody or an antibody fragment or derivative, a nucleic acid encoding an antibody or an antibody fragment or derivative, an inhibitory nucleic acid (antisense, siRNA, ribozyme, etc.) that inhibits protein synthesis, a peptide that inhibits TbKHC1 activity, or a molecule that specifically binds to the target molecules recognized by TbKHC1 in the host, in particular receptors that transmit the signal normally induced by TbKHC1 protein or components thereof, or a combination thereof.

In a particular embodiment, the inhibitor is a compound capable of specifically binding to TbKHC1 protein and neutralizing same. An example of such a compound is an antibody, or an antibody fragment or derivative, or an inhibitor conveyed by said antibody. The term “specific binding” refers to the fact that the specific inhibitor binds to TbKHC1 protein and does not specifically bind to other proteins or binds with much lower affinity (by a factor of 10 or more). Particularly preferably, the inhibitor is an antibody binding to TbKHC1 and not binding to endogenous proteins of the infected mammal.

The antibody may be a polyclonal antibody, a monoclonal antibody or an antibody fragment or derivative such as Fab or Fab′2 fragments, CDRs, single-chain antibodies (for example scFv), nanobodies, human or humanized antibodies, etc. The antibodies may be produced by techniques well-known to persons skilled in the art, for instance immunization of a non-human animal and collection of serum or antibody-producing cells. Monoclonal antibodies may be produced by obtaining hybridomas according to conventional techniques well-known to persons skilled in the art. By way of examples, antibodies according to the present invention may be generated by injecting TbKHC1 protein or an immunogenic peptide of the invention into animals (for example a rabbit or a mouse), then by collecting sera or B cells. The selectivity of the antibodies may then be tested and confirmed by conventional ELISA-type tests. Techniques for producing polyclonal or monoclonal antibodies, scFv fragments and human or humanized antibodies are described for example in Harlow et al., Antibodies: A Laboratory Manual, CSH Press, 1988; Ward et al., Nature 341 (1989) 544; Bird et al., Science 242 (1988) 423; WO94/02602; U.S. Pat. No. 5,223,409; U.S. Pat. No. 5,877,293; WO93/01288.

A particular object of the invention concerns an antibody specifically binding to TbKHC1 protein. More preferentially, the invention concerns an antibody binding to an epitope contained in the C-terminal region of TbKHC1 protein, for example an epitope contained in residues 687-1111 of TbKHC1 protein. The antibody of the invention is preferentially a monoclonal antibody.

Another object of the invention concerns an anti-TbKHC1 antibody able to be obtained by immunization of a non-human mammal with an immunogenic composition comprising a peptide comprising an epitope between residues 687 and 1111 inclusive of TbKHC1 protein.

Another object of the invention concerns a Fab or Fab′2 fragment of an antibody as defined above.

Another object of the invention concerns a single-chain anti-TbKHC1 antibody. It may be a nanobody, scFv, tandem antibody, etc.

Another inhibitor and object of the invention is a nucleic acid TbKHC1 inhibitor, notably an antisense nucleic acid, a ribozyme, or an interfering RNA specific for TbKHC1. Such nucleic acids comprise a portion (generally from 5 to 50 consecutive bases) of the coding sequence of TbKHC1 or the complementary strand thereof, for example a portion of sequence SEQ ID NO: 1 or the complementary strand thereof, and specifically inhibits expression (transcription or translation) of the protein.

Another inhibitor and particular object of the invention is a molecule inhibiting the effect of TbKHC1 in the mammalian host. It is in particular any molecule specifically binding to the target molecules recognized by TbKHC1 in the host, in particular host receptors that transmit the signal normally induced by TbKHC1 protein or components thereof. By way of example, mention may be made of sugars, peptides or other molecules (small drugs) that block TbKHC1 binding to cell or humoral receptors of the host.

### Veterinary and Pharmaceutical Compositions

The invention relates to any pharmaceutical or veterinary composition comprising (i) TbKHC1 protein, one or more antigenic peptides thereof, or an inhibitor of TbKHC1 protein, and (ii) a pharmaceutically or veterinarily acceptable excipient. Such compositions make it possible to block the action of TbKHC1 protein and thus to prevent or control trypanosome infection.

According to a first embodiment, the compositions of the invention are of vaccine type and induce a very powerful antiparasitic immunity in mammals. Thus, a particular object of the invention relates to compositions comprising (i) TbKHC1 protein, or one or more antigenic peptides thereof, (ii) a pharmaceutically or veterinarily acceptable excipient, and (iii) optionally an adjuvant. Such compositions make it possible to vaccinate or immunize mammals against TbKHC1 protein, and thus to protect the mammal against trypanosome infection or to treat such an infection.

The vaccines may comprise several antigenic peptides, so as to increase the immunogenicity of the vaccine. Thus, they may comprise several optionally-overlapping peptides each comprising from 5 to 100 amino acids and each comprising an amino acid sequence identical to a TbKHC1 protein domain comprised preferably between residues 687 and 1111. The vaccines may include other parasite molecules associated with TbKHC1.

In a particular embodiment, the vaccine comprises a single antigenic peptide.

In another particular embodiment, the vaccine comprises 2, 3, 4 or 5 separate antigenic peptides of TbKHC1. In this respect, a particular vaccine of the invention comprises antigenic peptides of TbKHC1 proteins from different strains of trypanosomes, thus increasing the potential of the vaccine. The vaccine may thus comprise one or more antigenic peptides of a TbKHC1 protein of one or more species of trypanosomes.

In another particular embodiment, the vaccine comprises an entire TbKHC1 protein.

In another particular embodiment, the vaccine comprises a nucleic acid encoding said TbKHC1 protein or the antigenic peptide(s).

In a particular embodiment, the composition according to the invention comprises the protein of sequence SEQ ID NO: 2 or a natural variant thereof, or a nucleic acid encoding said protein.

In a more particular embodiment, the composition according to the invention comprises a peptide comprising residues 1000 to 1111 of sequence SEQ ID NO: 2 or a natural variant thereof, a variant thereof with at least 90% sequence identity, or a nucleotide sequence encoding said peptide.

Advantageously, in the compositions of the invention, the protein or the antigenic peptide(s) or the nucleotide sequence is/are in pure, enriched extract, recombinant or synthetic form.

The veterinary vaccine compositions of the invention advantageously comprise an immunologically effective amount of TbKHC1 protein or antigenic peptides derived therefrom, as previously described, or a nucleic acid or an expression vector encoding or overexpressing TbKHC1 protein or antigenic peptide(s) thereof.

The vaccines according to the present invention may comprise one or more adjuvants so as to increase their efficacy. The adjuvants are well-known in the state of the art. By way of examples, mention may be made of aluminum salts in particular, such as aluminum hydroxide, metal salts, bacterial immunogens such as LPS, CT or LT, adjuvants of classes TLR3, TLR4, TLR5, TLR7, TLR8 or TLR9, saponins and derivatives thereof, oil-in-water or water-in-oil emulsions, polysaccharides, cationic liposomes, virosomes or polyelectrolytes. Other immunomodulators may be used, such as fly salivary proteins, cytokines or heat-shock proteins.

The vaccines according to the present invention may be monovalent vaccines (i.e., those that induce a response against a single type of pathogen) or multivalent vaccines (i.e., those capable of inducing a protective response against several distinct types of pathogens). In a particular embodiment, the invention thus aims at a multivalent vaccine comprising (i) TbKHC1 protein, or one or more antigenic fragments thereof, (ii) a pharmaceutically or veterinarily acceptable excipient, (iii) optionally an adjuvant and (iv) at least one antigen of another parasite. The vaccines according to the present invention may include several other parasite molecules, in combination with TbKHC1, notably other kinesins.

According to another embodiment, the compositions of the invention comprise an inhibitor of TbKHC1 protein and make it possible to treat, in a powerful and rapid manner, trypanosome infection in mammals. Thus, a particular object relates to a pharmaceutical or veterinary composition comprising (i) an inhibitor of TbKHC1 protein and (ii) a pharmaceutically or veterinarily acceptable excipient. Such compositions make it possible to block the action of TbKHC1 protein and thus to prevent or control infection with trypanosomes. In a preferred embodiment, the inhibitor is an anti-TbKHC1 antibody.

Thus, in a particular implementation of an embodiment of the invention, the invention relates to compositions characterized in that the inhibitor is an anti-TbKHC1 antibody, or a fragment or derivative of such an antibody.

In the compositions of the invention, any type of acceptable excipient may be used. In this respect, mention may be made of isotonic solutions, phosphate buffers or other saline solutions and culture media (for example physiological saline, PBS, Ringer lactate, medium 199, Ham's medium) and stabilizers and preservatives (for instance acids, sugars, phenoxyethanol, medium 199, albumin, amino acids and derivatives). Furthermore, the compositions of the invention may be in liquid or solid (powder) form. They may be packaged in any suitable container (ampule, syringe, phial, bottles, etc.).

As indicated, the compositions advantageously comprise an effective amount of TbKHC1 protein or antigenic peptide. This amount may be easily adapted by persons skilled in the art. Generally, the effective amount of TbKHC1 protein or peptide is an amount that induces an anti-TbKHC1 antibody response in the treated mammal. Such an amount is generally between 0.1 μg and 1 mg per dose, for example between 1 μg and 500 μg per dose, notably between 10 μg and 100 μg per dose.

In the case of a TbKHC1 inhibitor, the effective amount is an amount that inhibits by at least 10%, preferably by at least 20%, 30%, 40%, 50% or more, TbKHC1 production or activity in vitro or in vivo, in the treated mammal. Such an amount is generally between 0.1 μg and 1 mg of inhibitor per dose, preferably between 1 μg and 500 μg per dose.

The compositions of the invention may be used alone or in combination with other treatments, for instance trypanocides such as in particular pentamidine, eflornithine, nifurtimox, NECT, suramin, melarsoprol, fexinidazole, oxaborole, diminazene, isometamidium, homidium.

The invention may be used to treat any mammal potentially infected with a trypanosome, for instance cattle, sheep, cats, camels, dogs or humans. The compositions according to the present invention are particularly useful for treating pathologies induced by trypanosomes, such as in particular anemia, wasting and/or immunosuppression.

### Production of Antitrypanosomal Agents

The present invention also relates to a method for identifying, producing or optimizing antitrypanosomal compounds, comprising a step of evaluating the capacity of a test compound to inhibit the activity or production (or secretion) of TbKHC1 protein. Compounds endowed with such activity have a significant antitrypanosomal action.

The invention also relates to any compound identified, produced or optimized according to the preceding method, for use in the treatment of trypanosome infection.

### Diagnosis of Trypanosome Infection

TbKHC1 protein further constitutes a target of interest for detecting, in a mammal, the presence of trypanosomes. Said protein being secreted, it and any antibody against it (detection of antigen and/or antibody), or any nucleic acid encoding TbKHC1, can be detected in any fluid of the mammal, in particular the blood. Furthermore, the protein, like the antibodies, can make it possible not only to detect the presence of the parasite, but also to monitor the evolution of an infection and/or the efficacy of a treatment.

Thus, an object of the invention also concerns a method for in vitro diagnosis of trypanosomiasis or for detecting the presence of trypanosomes in a mammal, characterized in that it comprises measuring, in a sample from said mammal, or detecting the presence of, TbKHC1 protein or a nucleic acid encoding TbKHC1 protein or antibodies against TbKHC1.

An object of the invention also concerns a method for monitoring the evolution of trypanosome infection in a mammal, characterized in that it comprises measuring the amount of TbKHC1 protein or a nucleic acid encoding TbKHC1 protein or antibodies against TbKHC1 in samples from the mammal taken at various time intervals.

The invention also relates to a method for determining the efficacy of a treatment against trypanosomes in a mammal, characterized in that it comprises measuring the amount of TbKHC1 protein or a nucleic acid encoding TbKHC1 protein or antibodies against TbKHC1 in samples from the mammal taken at various time intervals during the treatment.

The invention also relates to a method for in vitro diagnosis of trypanosomiasis in a mammal, characterized in that it comprises measuring, in a sample from said mammal, the presence of TbKHC1 protein, a fragment thereof, a nucleotide sequence encoding TbKHC1, or antibodies against TbKHC1 or against one or more antigenic peptides thereof.

The invention further relates to a method for monitoring the evolution of trypanosome infection in a mammal, characterized in that it comprises measuring the amount of TbKHC1 protein, a fragment thereof, a nucleotide sequence encoding TbKHC1, or antibodies against TbKHC1, in samples from the mammal taken at various time intervals.

The invention also relates to a method for determining the efficacy of a treatment against trypanosomes in a mammal, characterized in that it comprises measuring the amount of TbKHC1 protein, a fragment thereof, a nucleotide sequence encoding TbKHC1, or antibodies against TbKHC1, in samples from the mammal taken at various time intervals during the treatment.

The presence or the relative amount of TbKHC1 protein or antibody may be determined by any technique known per se, such as in particular by means of a specific ligand, for example an antibody or an antibody fragment or derivative, or the protein or a fragment thereof or an epitope or a mimotope. Preferably, the ligand is an antibody specific for the polypeptide, or a fragment of such an antibody (for example Fab, Fab′, CDR, etc.), or a derivative of such an antibody (for example a single-chain antibody, scFv), or the protein or a fragment thereof or an epitope or a mimotope. The ligand is typically immobilized on a support, such as a slide, bead, column, plate, etc. The presence or the amount of protein of interest or of fragments thereof or of antibody in the sample may be detected by visualizing a complex between the target and the ligand, for example by using a labeled ligand, by using a second labeled visualization ligand, etc. Well-known immunological techniques which may be used include ELISA, RIA, etc. If necessary, the amount of polypeptide detected can be compared with a reference value, for example a median or mean value observed among human patients or non-human mammals which are not infected, or with a value measured in parallel in a control sample.

All immunological techniques based on antigen-antibody reactions may be employed, using either TbKHC1 protein or fragments thereof or natural or synthetic derivative compounds or an epitope, a mimotope as antigen, or molecules specifically recognizing TbKHC1 protein or fragments thereof or natural or synthetic derivative compounds (for example, antibody, Fab or Fab′ fragments, CDR, or derivatives of an antibody or a nanobody). Basic immunological techniques, for example agglutination, precipitation, immunoenzymatic techniques, immunoblotting, Western blot, are suitable.

In another variant, the invention detects the presence of nucleic acid encoding TbKHC1. This detection may be carried out by techniques known per se to persons skilled in the art, such as in particular by Northern blot, selective hybridization, use of supports coated with oligonucleotide probes, selective amplification of nucleic acid, for instance by RT-PCR, quantitative PCR or ligation-PCR, etc. These methods may include the use of a nucleic probe (for example an oligonucleotide) capable of selectively or specifically detecting the target nucleic acid in the sample. Amplification may be carried out according to various methods known per se to persons skilled in the art, such as PCR, LCR, transcription-mediated amplification (TMA), strand-displacement amplification (SDA), NASBA, use of allele-specific oligonucleotides (ASO), allele-specific amplification, loop-mediated isothermal amplification (LAMP), recombinase polymerase amplification (RPA), Southern blot, single-strand conformation analysis (SSCA), in situ hybridization (e.g., FISH), gel migration, heteroduplex analysis, etc.

According to a preferred implementation of an embodiment, the method comprises detecting the presence or the absence (or the relative amount) of a nucleic acid encoding TbKHC1 by selective hybridization or selective amplification.

Selective hybridization is typically carried out by using nucleic probes, preferably immobilized on a support, such as a solid or semi-solid support having at least one surface, planar or not, on which nucleic probes can be immobilized. Examples of such supports include a slide, bead, membrane, filter, column, plate, etc. They may be made of any compatible material, such as in particular glass, silica, plastic, fiber, metal, polymer, etc. The nucleic probes may be any nucleic acid (DNA, RNA, PNA, etc.), preferably single-stranded, comprising a sequence specific for a nucleic acid encoding TbKHC1. The probes typically comprise from 5 to 400 bases, preferably from 8 to 200, more preferentially fewer than 100, and even more preferentially fewer than 75, 60, 50, 40 or even 30 bases. The probes may be synthetic oligonucleotides, produced on the basis of sequence SEQ ID NO: 1 according to conventional synthesis techniques. Such oligonucleotide probes typically comprise from 10 to 50 bases, preferably from 20 to 40, for example around 25 bases. The probes may be synthesized beforehand and then deposited on the support, or synthesized directly in situ, on the support, according to methods known per se to persons skilled in the art. The probes may also be manufactured by genetic techniques, for example by amplification, recombination, ligation, etc.

The probes thus defined constitute another object of the present application, as well as uses of same (primarily in vitro) for detecting trypanosome infection in a subject.

Hybridization may be carried out under conventional conditions known to and adjustable by persons skilled in the art (Sambrook, Fritsch, Maniatis (1989) Molecular Cloning, Cold Spring Harbor Laboratory Press). In particular, hybridization may be carried out under conditions of high, moderate or low stringency, depending on the desired level of sensitivity, the amount of material available, etc. For example, suitable hybridization conditions include a temperature of between 55° C. and 63° C. for 2 to 18 hours. Other hybridization conditions, suitable for high-density supports, are for example a hybridization temperature of between 45° C. and 55° C. After hybridization, various washes may be carried out to eliminate unhybridized molecules, typically in SSC buffers containing SDS, such as a buffer containing 0.1× to 10×SSC and 0.5% to 0.01% SDS. Other wash buffers containing SSPE, MES, NaCl or EDTA may also be used.

A particular object of the invention thus concerns a method for detecting the presence of a trypanosome in a mammal, or for evaluating the response to a treatment against trypanosomes, comprising contacting, under conditions allowing hybridization between complementary sequences, nucleic acids from a sample from the mammal and a nucleic probe specific for TbKHC1, the formation of a hybrid being indicative of the presence of trypanosomes.

Selective amplification is preferably carried out by using a primer or a primer pair allowing amplification of all or part of a nucleic acid encoding TbKHC1. The primer may be specific for a coding sequence (for example SEQ ID NO: 1), or for a region flanking the coding sequence. The primer typically consists of a single-stranded nucleic acid having a length advantageously of between 5 and 50 bases, preferably between 5 and 30. Such a primer constitutes another object of the present application, as well as the use thereof (primarily in vitro) for detecting the presence of trypanosomes in a subject.

In this respect, another object of the invention concerns the use of a nucleotide primer or of a set of nucleotide primers allowing amplification of all or part of a TbKHC1 gene for detecting the presence of trypanosomes in a mammal.

Another particular object of the invention concerns a method for detecting the presence of trypanosomes in a mammal, comprising contacting, under conditions allowing amplification, nucleic acids from a sample from the mammal and a primer specific for TbKHC1, the existence of an amplification product being characteristic of the presence of trypanosomes in said mammal.

The detection method can be applied to any biological sample from the mammal being tested. As such, the term “sample” generally refers, within the meaning of the invention, to any sample containing nucleic acids or polypeptides. Mention may be made advantageously of a sample of blood, plasma, platelets, ganglion, saliva, urine, stool, etc., more generally any tissue, organ or, advantageously, biological fluid containing nucleic acids or polypeptides or antibodies. In a preferred and particularly advantageous embodiment, the sample used for the detection method is a sample derived from blood, for example a sample of blood, serum or plasma. The sample may be obtained by any technique known per se, for example by sampling, by non-invasive techniques, from sample collections or libraries, etc. The sample may also be pretreated to facilitate the accessibility of the protein or the nucleic acid thereof, for example by lysis (mechanical, chemical, enzymatic, etc.), purification, centrifugation, separation, etc. The sample may also be labeled, to facilitate detecting the presence of the target molecules (fluorescent, radioactive, luminescent, chemical or enzymatic labeling, etc.). The nucleic acids of the sample may also be separated, treated, enriched, purified, reverse-transcribed, amplified, fragmented, etc. In a particular embodiment, the nucleic acids of the sample are DNA or RNA, notably mRNA of the sample. In a more particular embodiment, the nucleic acids are the amplification product of RNA, notably of mRNA; or cDNA prepared from RNA, notably mRNA of the sample.

The presence of TbKHC1 protein (or a nucleic acid encoding the protein) in the sample is indicative of the presence of trypanosomes in the mammal concerned.

### Kits

Another object of the invention relates to a kit for detecting or measuring trypanosomes in a test sample, characterized in that it comprises at least one ligand specific for TbKHC1 protein, and at least one reagent for detecting a reaction between the ligand and TbKHC1 protein. Advantageously, the ligand is an anti-TbKHC1 antibody and the reagent allows detection of an immune complex. The kit may comprise a suitable support (for example a plate, column, chip, etc.) on which the ligand is immobilized, allowing easy detection of complex formation. The detection reagent may be a second ligand (e.g., antibody) binding to TbKHC1 protein or binding to the first ligand. It may be any other reagent making it possible to reveal complex formation (enzyme, stain, etc.).

Another object of the invention relates to a kit for detecting or measuring trypanosomes in a test sample, characterized in that it comprises at least one ligand specific for an anti-TbKHC1 antibody, and at least one reagent for detecting a reaction between the ligand and the antibody. Advantageously, the ligand is a TbKHC1 protein or an antigenic peptide of TbKHC1, or a synthetic product, and the reagent allows detection of an immune complex. The kit may comprise a suitable support (for example a plate, column, chip, etc.) on which the ligand is immobilized, allowing easy detection of complex formation. The detection reagent may be a second ligand (e.g., antibody) binding to the antibodies. It may be any other reagent making it possible to reveal complex formation (enzyme, stain, etc.).

Another object relates to a kit for detecting or measuring trypanosomes in a test sample, characterized in that it comprises at least one antibody according to claim 12 or TbKHC1 protein or a peptide thereof, a medium suitable for the formation of an immune complex, and at least one reagent for detecting an immunological reaction.

Another object relates to a kit for detecting or measuring trypanosomes in a test sample, characterized in that it comprises at least one nucleic probe specific for TbKHC1, a medium suitable for hybridization, and at least one reagent for detecting a hybridization reaction.

Another object of the present application relates to a product comprising a support on which at least one ligand specific for TbKHC1 protein is immobilized. Preferably, the ligand is an antibody or a fragment or derivative of anti-TbKHC1 antibody.

Another object of the present application relates to a product comprising a support on which at least one TbKHC1 protein or an antigenic peptide thereof is immobilized.

Another object of the present application relates to a product comprising a support on which at least one nucleic probe specific for TbKHC1 is immobilized. Preferably, the nucleic probe is a single-stranded DNA molecule of 10 to 200 nucleotides in length, having a sequence complementary to the gene encoding TbKHC1 protein.

The support may be any solid or semi-solid support having at least one surface, planar or not (i.e., in 2 or 3 dimensions), allowing the immobilization of nucleic acids or polypeptides. Such supports are for example a slide, bead, membrane, filter, column, plate, etc. They may be made of any compatible material, such as in particular glass, silica, plastic, fiber, metal, polymer, polystyrene, Teflon, etc. The reagents may be immobilized on the surface of the support by known techniques or, in the case of nucleic acids, synthesized directly in situ on the support. Immobilization techniques include passive adsorption (Inouye et al., J. Clin. Microbiol. 28 (1990) 1469), covalent bonding. Techniques are described for example in WO90/03382, WO99/46403. The reagents immobilized on the support may be arranged according to a preestablished plan, to facilitate detecting and identifying the complexes formed, and according to a variable and adaptable density. The products of the invention typically comprise control molecules for calibrating and/or standardizing the results.

Other aspects and advantages of the invention will appear upon reading the following examples, which should be regarded as illustrative and non-limiting.

### EXAMPLES

### Materials & Methods

### Animals

Female Swiss mice weighing 25-30 g (Charles River, Domaine des Oncins, 69592 L'Arbresle Cedex) maintained in the laboratory in accordance with animal welfare regulations.

### Parasites

The following parasite strains were used:

Trypanosoma brucei brucei (Antat 1.1 E)
Trypanosoma brucei gambiense “Feo” (ITMAP 1893)
Trypanosoma brucei gambiense “Biyamina” (MHOM/SD 82),
Trypanosoma brucei brucei EATRO 1125
Trypanosoma musculi “Partinico II”
Trypanosoma brucei rhodesiense (Etat 1.2/R)
Trypanosoma evansi (Mantecal EC8)
Trypanosoma congolense (E325)
Trypanosoma cruzi (MN cl2)

### Preparation of the Secretome

The parasites are purified by ion-exchange chromatography (DEAE cellulose) from the blood of infected mice and incubated for 2 hours in secretion medium (Ringer lactate+50 mM glucose) at 37° C. The secretory products are collected by centrifugation (1200 g, 10 minutes, 4° C.). This supernatant is filtered on a 0.22 m filter, aliquoted and stored at −80° C. The amount of proteins present is measured by the Bradford method. This secretome, also called parasite soluble factor (PSF) or secretory product, containing TbKHC1 protein, may be used as such.

### Production of an Anti-TbKHC1 Antibody

BALB/c mice were immunized with a preparation containing TbKHC1 protein to produce hybridomas by fusion. The resulting monoclonal antibodies were used to screen an expression library for T. b. gambiense. Recognized clones were then used to produce recombinants. The monoclonal antibody selected, Mab1, binds to the C-terminal region of kinesin, a region predicted to be a coiled region.

### Preparation of a Fraction Enriched in TbKHC1 Protein by Affinity Chromatography

The parasites are purified by ion-exchange chromatography (DEAE cellulose) from the blood of infected mice and incubated for 2 hours in secretion medium (Ringer lactate+50 mM glucose) at 37° C. The secretory products (secretome) are collected by centrifugation (1200 g, 10 minutes, 4° C.). This supernatant is filtered on a 0.22 m filter, aliquoted and stored at −80° C. The amount of proteins present is measured by the Bradford method.

Monoclonal antibody Mab1 in buffer solution (0.1 M carbonate/5 M NaCl, pH 8.3) is grafted onto a chromatography column (Sephadex CNBr). After 48 hours at 4° C. and 5 washes with carbonate buffer, the secretory products are deposited on this column. After passage and successive washes with secretion medium, the molecules bound to the antibody are eluted by successively adding 1 M glycine buffer/HCl (pH 3) and 1 M glycine buffer/NaOH (pH 11). The pH of the eluted fraction is adjusted to pH 8. After dialysis in 0.015 M PBS, the enriched fraction is aliquoted into tubes stored at −80° C. The amount obtained is measured according to the Bradford method. The contents of the enriched fraction are analyzed by gel electrophoresis and Western blot.

### Preparation of a Fraction Enriched in TbKHC1 Protein by Differential Filtration

In this example, vaccine fractions were prepared from secretory products by differential filtration. The parasites were purified on an ion-exchange column (DEAE cellulose) and placed under secretory conditions (200×10⁶ per mL of secretion medium for 2 hours). The secretory products (PSF) of the following strains of trypanosome species were thus obtained:
T. brucei brucei (T. b. b);
T. brucei brucei KO for both TbKHC1 alleles (T. b. b KO);
T. brucei gambiense (T. Feo);
T. evansi (T. e).

The PSF were then fractionated by differential filtration allowing separation into high molecular weights (HMW) and low molecular weights (LMW). Various cut-offs were used for the filtrations: 50 kDa, which gives fractions HMW 50 and LMW 50; and 100 kDa, which gives fractions HMW 100 and LMW 100. The intermediate fraction results from passing the PSF first through the filter having the 50 kDa cut-off and then through the filter having a 100 kDa cut-off, and corresponds to the molecules having a MW>50 kDa, but <100 kDa. The secretome is placed on a filter having a 50 kDa or 100 kDa cut-off, after centrifugation (4000 g, 1 hour, 4° C.), the fraction containing molecules having a molecular weight lower than the cut-off (LMW, low molecular weight) is separated from that containing molecules having a molecular weight higher than the cut-off (HMW, high molecular weight). TbKHC1 protein is present in the HMW fractions. The fractions are aliquoted into tubes stored at −80° C. The amount obtained in each fraction is measured according to the Bradford method.

### Example 1: Inhibition of Parasite Growth by a Monoclonal Antibody Against Kinesin

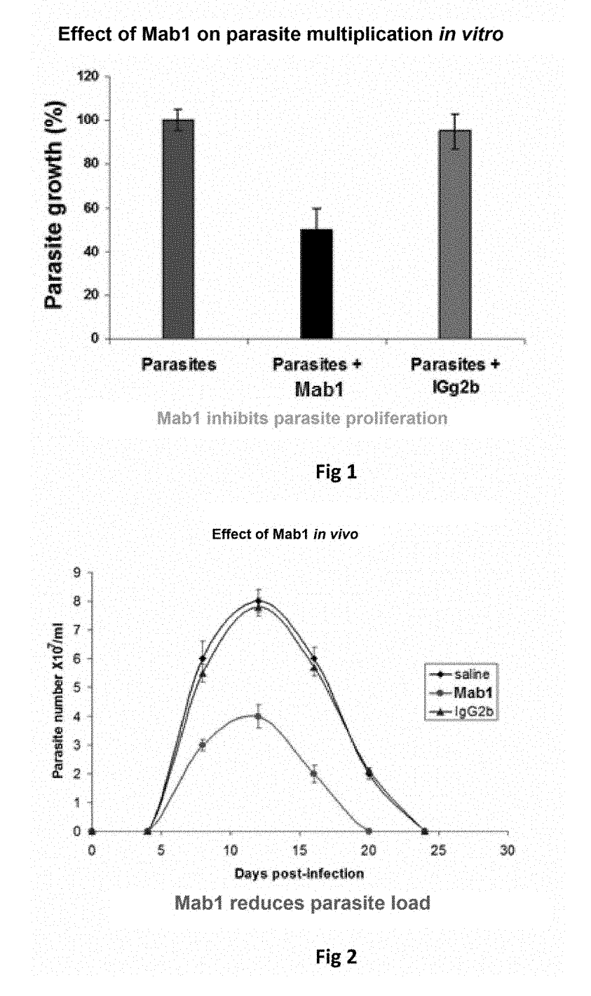

In vitro, monoclonal antibody Mab1 was added to parasites in co-culture with feeder layers (Mab1 concentration in the culture: 4 μg/mL). Compared with the control, Mab1 inhibits parasite growth whereas the IgG2b isotype control (concentration: 4 μg/mL) has no effect (FIG. 1).

In vivo, injecting Mab1 (200 μg in 200 μL of PBS intraperitoneally) into mice parasitized for 2 days inhibits the development of parasitemia (expressed in log 10 of parasite number per mL of blood); the IgG2b isotype control (200 μg in 200 μL of PBS intraperitoneally) has no effect (FIG. 2).

### Example 2: In Vivo Protection Against Parasites by Vaccination

The fraction enriched in TbKHC1 protein is injected (10-20 μg/mouse) twice, with a 30-day interval (D0 and D30), into the mouse via the subcutaneous route, with or without adjuvant (saponin, 25 μg per mouse). Control mice receive medium alone with or without adjuvant.

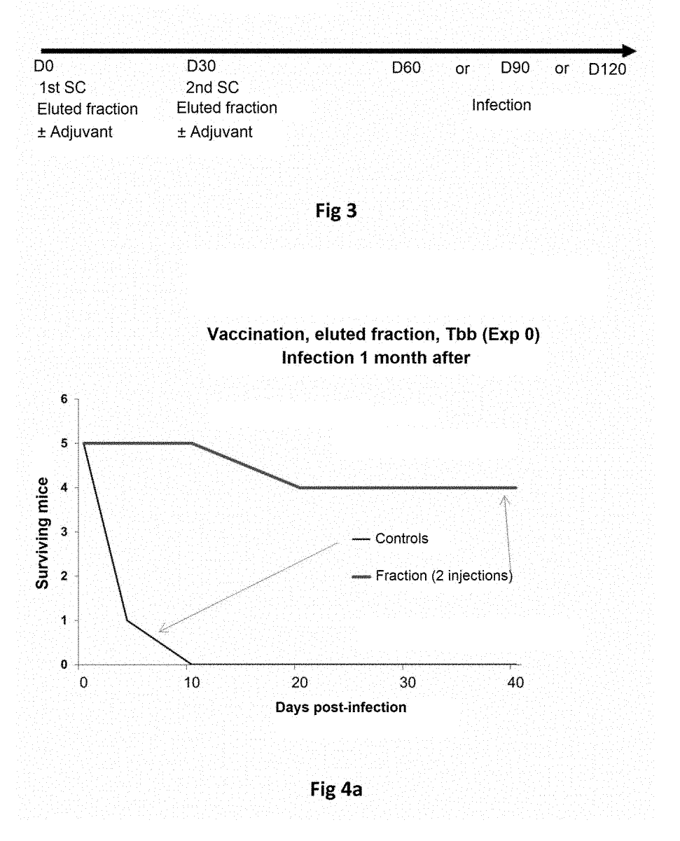

The mice are infected 1, 2 or 3 months after the last injection (see FIG. 3).

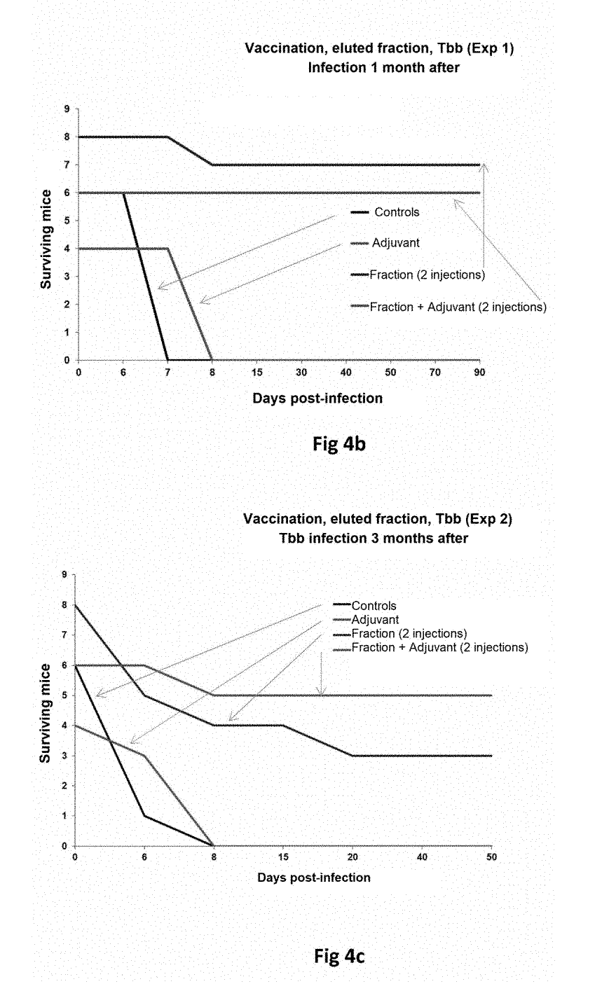

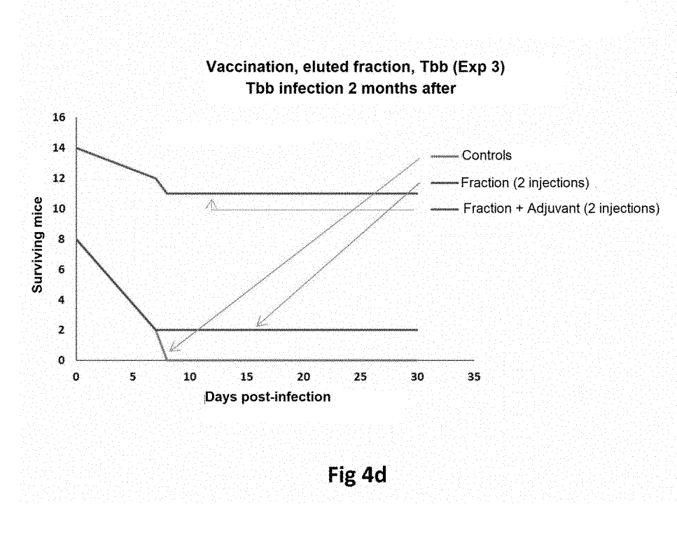

The parasitemia of the vaccinated mice and of the controls (medium alone±adjuvant) is evaluated daily for 25 days post-infection, then once per week thereafter. The results are presented in FIG. 4.

Control mice having received the medium with or without adjuvant die around the 7^{th}-8^{th} day post-infection. Remarkably, 22 of 26 mice (84.6%) having received two injections of TbKHC1 protein+adjuvant survive. Adding the adjuvant to TbKHC1 protein increases the survival rate of vaccinated mice and the duration of efficacy of the vaccine.

### Example 3: Vaccination with TbKHC1 Induces Cross-Protection

Mice are immunized with TbKHC1 protein and then infected 2 months after either with the same trypanosome or with a trypanosome of another species.

| | | | Infection 2 | |
|---|---|---|---|---|
| | | Number of | months after | |
| | Origin of | subcutaneous | the last | |
| | TbKHC1 for | injections + | injection + | Parasitemia |
| Batches | immunization | saponin | adjuvant | and survival |
| Batch | T. brucei | 2 | T. b. | No parasite |
| 1 (14 | gambiense | | gambiense | detected in the |
| mice) | | | | mice; all |
| | | | | survive at 50 |
| | | | | days |
| Batch | T. brucei | 2 | T. b. brucei | No parasite |
| 2 (14 | gambiense | | | detected in the |
| mice) | | | | mice; all |
| | | | | survive at 50 |
| | | | | days |

These results show cross-protection, wherein TbKHC1 protein of T. brucei gambiense is capable of inducing protection against infection with T. brucei brucei.

### Example 4: Diagnosis of Infected Patients

Presence of anti-TbKHC1 antibody in the serum of patients with human African trypanosomiasis and absence of same in the serum of control subjects from the same endemic area.

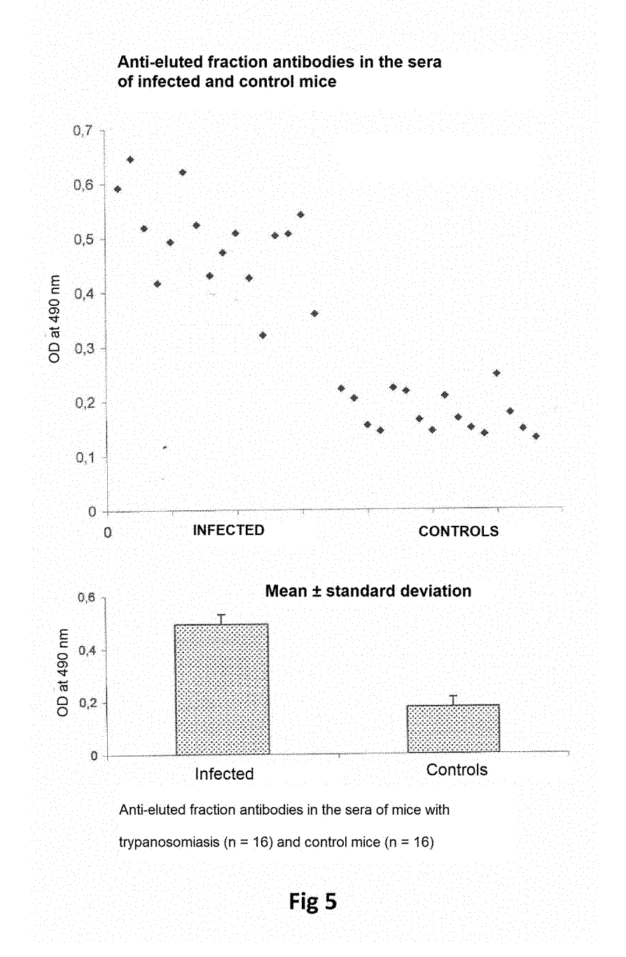

The results are presented in FIG. 5. They show that TbKHC1 anti-kinesin antibodies were detected in all human African trypanosomiasis patients tested. These results thus illustrate the possibility of distinguishing infected patients by measuring antibodies against TbKHC1.

### Example 5: Test of Protection by Serotherapy

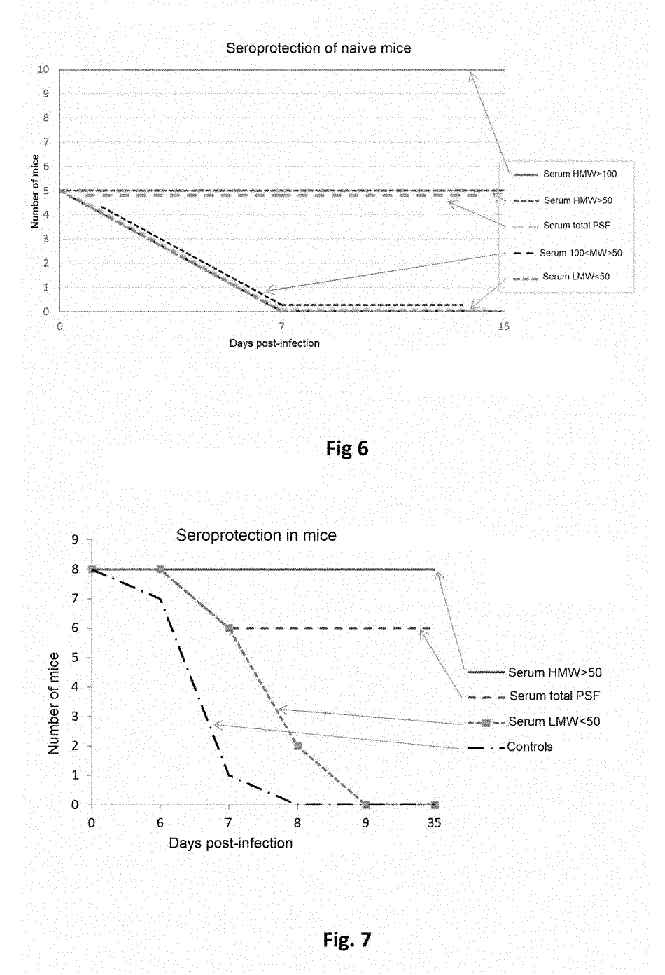

Serotherapy tests carried out on batches of 5, 8 or 10 mice showed that sera from mice having received two injections 3 weeks apart of total PSF of T. Feo (30 μg/injection), or the HMW 50 (20 μg/injection) or HMW 100 (20 μg/injection) fraction, in the presence of saponin (25 μg), effectively protect (100% protection) naive mice experimentally infected with T. Feo (2000 parasites subcutaneously) (FIG. 6). On the other hand, sera from mice having received two injections 1 month apart of the LMW50 or intermediate (100<MW>50) fraction have no protective effect in infected naive mice. Mice receiving normal mouse serum before infection (not shown in FIG. 6) die 7 days post-infection.

Moreover, mice having received serum from mice immunized with PSF of T. Feo or with the HMW50 fraction of T. Feo are also protected against infection with T. b. b (cross-protection) (FIG. 7).

### Example 6: Test of Protection by Vaccination

6.1. Vaccination with a T. b. Gambiense (Feo) Fraction

Naive mice receive two injections with a 3-week interval, in the presence of saponin (25 μg/mouse) and fractions derived from T. b. gambiense (Feo), namely total PSF (30 μg/injection), HMW50 (20 μg/injection) or LMW50 (20 μg/injection). The mice are then challenged with living T. b. b parasites (2000 per mouse) 2 months after administration of the second immunization.

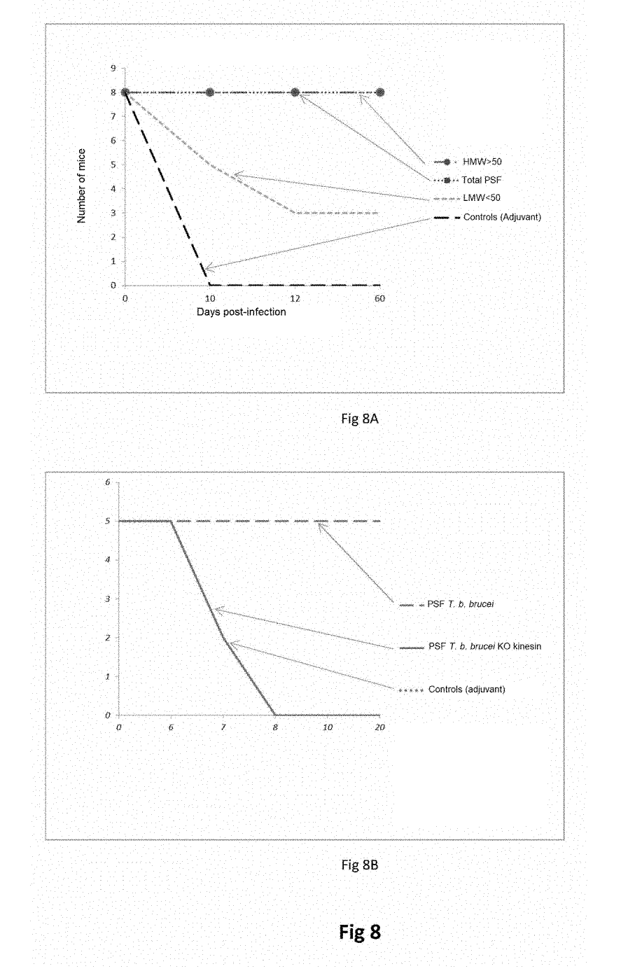

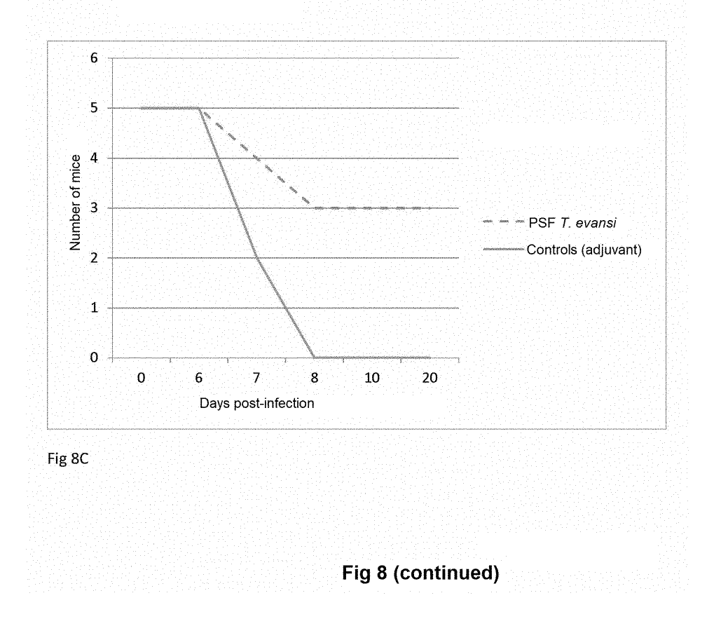

The results presented in FIG. 8A show:
that the protective antigens are present mainly in the high molecular weights of PSF of T. Feo; and
that cross-protection is obtained against infection with T. b. brucei.
   6.2. Vaccination with a T. b. Brucei Fraction

Mice receive two injections of total PSF (50 μg) of T. b. brucei or of T. b. brucei KO for kinesin with a 30-day interval (D0 and D30) subcutaneously with adjuvant (saponin, 25 μg per mouse). “Control” mice receive adjuvant alone. The mice are infected 2 months after the last injection (D30) with 2000 living T. b. brucei parasites.

The results presented in FIG. 8B show that all mice having received PSF of T. b. brucei survive, whereas mice having received PSF of T. b. brucei KO for kinesin died 8 days post-infection, at the same time as the “control” mice.

6.3. Vaccination with a T. evansi Fraction

Mice receive two injections of total PSF (50 μg) of T. evansi, with a 30-day interval (D0 and D30), via the subcutaneous route with adjuvant (saponin, 25 μg per mouse). “Control” mice receive adjuvant alone. The mice are infected 2 months after the last injection with T. evansi (2000 parasites).

The results presented in FIG. 8C show enhanced survival of mice having received PSF of T. evansi, whereas all the controls die.

### Example 7: Proteomic Analyses

### 7.1. Protein Profiles

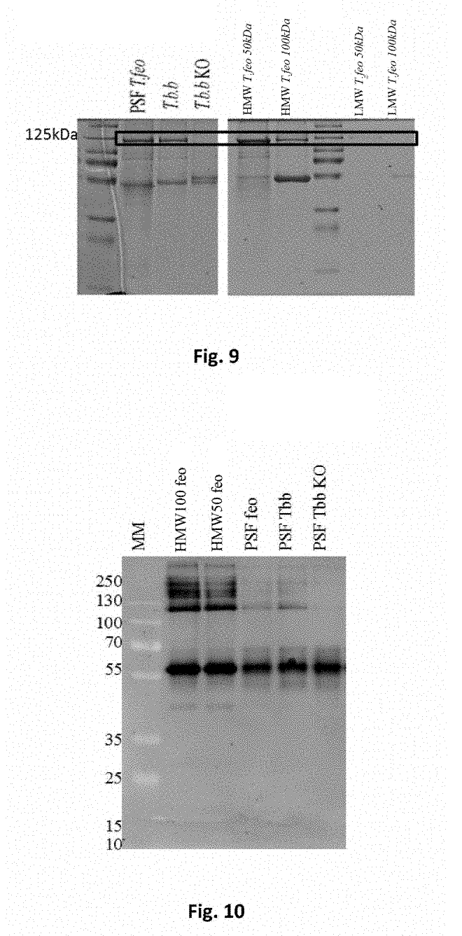

Protein profiles, obtained after migration under denaturing and non-reducing conditions, of the secretome or of secretome fractions (HMW or LMW), show the presence of a high molecular weight band (around 125 kDa, boxed region in FIG. 9) sufficiently present in pathogenic species of trypanosomes (T. Feo; T. b. brucei; T. rhodesiense; T. evansi) to be detected after Coomassie blue staining. This band corresponds to the molecular weight of TbKHC1 protein. On the other hand, this band appears to be absent, or in too small an amount, in T. b. brucei KO for kinesin. Significantly, this band is indeed present in the HMW fractions and is poorly visualized in the LMW fractions.

### 7.2. Mass Spectrometry

All samples were analyzed by nanoflow HPLC (Ultimate 3000, Dionex) coupled to a mass spectrometer with a nanoelectrospray source (Orbitrap Elite, Thermo Fisher Scientific). The peptides were separated on a capillary column (C18 reverse-phase, NanoViper, Dionex) according to a 0-40% gradient of B over 60 min (105-minute run) (A=0.1% formic acid, 2% acetonitrile; B=0.1% formic acid in acetonitrile) with a flow rate of 300 nL/min. Spectra were recorded via the Xcalibur software (Thermo Fisher Scientific). Spectral data were analyzed via the MaxQuant 1.5.0.0 software and then reprocessed with the Perseus 1.5.3.0 software after applying the Leading v2.2 script developed by Oana Vigy. The database we used was: Uniprot_Trypanosoma-all_2016_01.fasta with the following modifications: Carbamidomethylation (C) in fixed mode and Oxidation (M) in variable mode.

This technique made it possible to identify the presence of TbKHC1 in the protective samples of T. Feo (PSF Feo and HMW50) and the absence of same in the LMW 50 fraction.

### 7.3. Analysis of the Immunological Profile by Western Blot

After differential filtration of the secretory products (PSF), the protective antigens are concentrated in the HMW50 and HMW100 fractions. The sera from mice immunized with these fractions and protected were used to analyze the antigenic targets of the protective antibodies. Molecular weight markers (MM) were used to estimate the molecular weight of the antigens revealed by the sera.

Western blot developed with purified Mab1 (FIG. 10):

Purified monoclonal antibody Mab1 targets TbKHC1 kinesin. It recognizes high molecular weight proteins and notably a protein with an apparent molecular weight of about 125 kDa and a 59 kDa protein that appears to be common to all the trypanosome species studied. Our data show that the 125 kDa antigen corresponds to TbKHC1 kinesin (125.89 kDa). The 59 kDa protein corresponds to a protein fragment. This recognition for the 125 kDa protein is very high in the HMW 100 Feo and HMW50 Feo samples; less so for the PSF Feo, PSF T. b. brucei samples; and low or even non-existent for the PSF T. b. brucei KO samples.

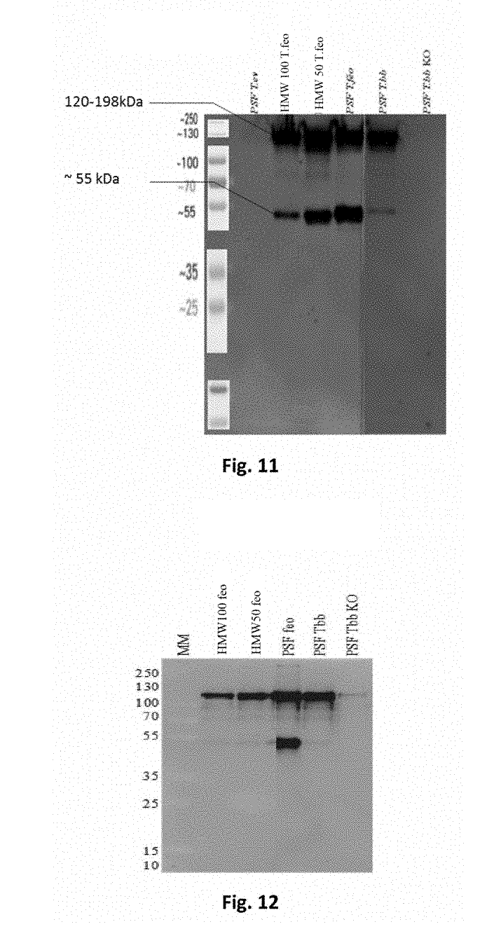

Western blot developed with anti-PSF Feo serum (FIG. 11):

Two major immunogenic complexes, between 198 kDa and 120 kDa and around 55 kDa, are revealed by the anti-PSF Feo serum.

Western blot developed with anti-HMW50 Feo serum (FIG. 12):

The anti-HMW 50 serum selectively targets the antigen corresponding to a molecular weight of 125 kDa: Differential filtration >50 thus concentrates this antigen, which corresponds to TbKHC1 kinesin protein (125.89 kDa). The 125 kDa antigen is virtually unrecognized by this serum in PSF T. b. brucei KO for TbKHC1. That confirms that the 125 kDa antigen corresponds to TbKHC1 kinesin.

Western blot developed with anti-HMW 100 Feo serum:

The anti-HMW 100 serum also preferentially targets a 125 kDa antigen: Differential filtration >100 concentrates this antigen corresponding to TbKHC1 kinesin protein (125.89 kDa).

1-17. (canceled)

18. A pharmaceutical or veterinary composition comprising (i) TbKHC1 protein or one or more antigenic peptides thereof; a nucleic acid encoding said protein or said peptide; or an inhibitor of TbKHC1 protein, and (ii) a pharmaceutically or veterinarily acceptable excipient.

19. The composition according to claim 18, which comprises (i) TbKHC1 protein or one or more antigenic peptides thereof, or a nucleic acid encoding said protein or said peptide, (ii) a pharmaceutically or veterinarily acceptable excipient, and (iii) optionally an adjuvant.

20. The composition according to claim 18, which comprises the TbKHC1 protein of sequence SEQ ID NO: 2 or a natural variant thereof, or a nucleic acid encoding said protein.

21. The composition according to claim 18, which comprises a peptide comprising residues 1000 to 1111 of sequence SEQ ID NO: 2 or of a natural variant thereof, a variant thereof with at least 90% sequence identity, or a nucleotide sequence encoding said peptide.

22. The composition according to claim 18, wherein the protein or the antigenic peptide(s) or the nucleotide sequence is/are in pure, enriched extract, recombinant or synthetic form.

23. The composition according to claim 18, wherein the inhibitor is an anti-TbKHC1 antibody, or a fragment or derivative of such an antibody.

24. A method for vaccinating or immunizing a mammal against trypanosomes, comprising administering to the mammal a TbKHC1 protein, or one or more antigenic peptides thereof, or a nucleic acid encoding said protein or said peptide, or a secretion extract enriched in said protein.

25. The method according to claim 24, for protecting a mammal against trypanosomiasis.

26. The method according to claim 24, for treating a mammal with trypanosomiasis.

27. The method according to claim 24, which comprises administering TbKHC1 or a peptide comprising residues 1000 to 1111 of sequence SEQ ID NO: 2 or of a natural variant thereof or a variant thereof with at least 90% sequence identity.

28. A method for treating a mammal with trypanosomiasis, comprising administering to said mammal an inhibitor of TbKHC1 protein.

29. The method according to claim 28, wherein the inhibitor is an antibody specifically binding to TbKHC1 protein.

30. A method for in vitro detection of trypanosomiasis in a mammal, comprising measuring, in a sample from said mammal, the presence of TbKHC1 protein, a fragment thereof, a nucleotide sequence encoding TbKHC1, or antibodies against TbKHC1 or against one or more antigenic peptides thereof, said presence being indicative of trypanosomiasis.

31. The method according to claim 30, said method monitoring the evolution of trypanosome infection in a mammal and comprising measuring the amount of TbKHC1 protein, a fragment thereof, a nucleotide sequence encoding TbKHC1, or antibodies against TbKHC1, in samples from the mammal taken at various time intervals.

32. The method according to claim 30, said method comprising determining the efficacy of a treatment against a trypanosome infection by measuring the amount of TbKHC1 protein, a fragment thereof, a nucleotide sequence encoding TbKHC1, or antibodies against TbKHC1, in samples from the mammal taken at various time intervals during the treatment.

33. A kit for detecting or measuring trypanosomes in a test sample, characterized in that it comprises:
(a)(i) at least one antibody specifically binding to TbKHC1 protein, or a TbKHC1 protein, or a peptide thereof, (ii) a medium suitable for the formation of an immune complex, and (iii) at least one reagent for detecting an immunological reaction; or
(b) at least one nucleic probe specific for TbKHC1, a medium suitable for hybridization, and at least one reagent for detecting a hybridization reaction.
